(19) United States
(12) Patent Application Publication
(10) Pub. No.: US 2026/0298945 A1
(43) Pub. Date: Oct. 1, 2026

(54) PREDICTING NON-RESPONSIVENESS OF IBD PATIENTS

(71) Applicant: NAVOLAB DIAGNOSZTIKA KFT, Szeged (HU)

(72) Inventors: Balázs HALLGAS, Szeged (HU); Tamás MOLNÁR, Szeged (HU)

(21) Appl. No.: 19/479,325

(22) PCT Filed: Apr. 29, 2024
(86) PCT No.: PCT/HU2024/050031

§ 371 Date: Oct. 27, 2025

(30) Foreign Application Priority Data
Apr. 28, 2023 (HU) P2300141

(51) Int. Cl. G01N 33/68

(52) U.S. Cl. CPC G01N 33/6893; G01N 2333/47; G01N 2800/065; G01N 2800/52

(57) ABSTRACT

Predicting non-responsiveness of IBD patients The present invention relates to an in vitro method for predicting the responsiveness of an IBD patient to a therapy with an intracellularly acting immunosuppressive agent of interest, wherein a sample is provided from the IBD patient at an initial period of a treatment with the immunosuppressive agent of interest, said sample comprising effector mononuclear cells, and responsiveness is predicted from the difference between a multidrug ABC transporter activity level in the effector mononuclear cells in said sample and a reference transporter activity level. The method is useful in a treatment of IBD, e.g. in monitoring the progress of the disease or in a decision on a shift from an initial treatment with an agent to another agent like csDMARD or tsDMARD.

### FIELD OF THE INVENTION

The present inventors have found that responsiveness of an IBD patient to an intracellularly acting immunosuppressive agent can be assessed by measuring an ABC-transporter activity level, preferably transport activity level. The invention relates to a method for predicting the responsiveness of an IBD patient to a therapy with an intracellularly acting immunosuppressive agent of interest, from a biological sample taken from said IBD patient at a time before treatment with the immunosuppressive agent of interest or up to the 4th month of treatment with the immunosuppressive agent of interest, said sample comprising effector mononuclear cells, by the measurement of transport activity by a multidrug ABC transporter.

### BACKGROUND OF THE INVENTION

### Inflammatory Bowel Disease

Crohn's disease (CD) and ulcerative colitis (UC), together inflammatory bowel disease (IBD), are chronic relapsing and remitting disorders that impose a life-long burden on an increasingly large portion of the population. At the turn of the 21st century, inflammatory bowel disease has become a global disease with accelerating incidence in newly industrialised countries whose societies have become more westernised. Although incidence is stabilising in western countries, burden remains high as prevalence surpasses 0.3%. Health care costs after a patient has developed CD or UC are high [Ng S C et al., 2017]. CD affects almost every part of the gastrointestinal tract and tissue layers, and the most frequent locations are the terminal ileum and colon. However, UC mainly involves the superficial intestinal mucosa of the colon, including the proximal end of the rectum [Torres, J et al., 2017, Ungaro, R. et al., 2017, D'Haens G et al., 2008].

The pathogenesis of IBD is still not clearly understood despite intensive research, but an inappropriate mucosal immune response to gut microflora is a well-recognized phenomenon in the pathogenesis [Xu X R et al., 2014, Lamas B et al., 2016]. Although displaying different clinical features, both major subcategories of inflammatory bowel disease (IBD), UC and CD are characterized by infiltration of the gut wall by inflammatory effector cells, elevated biomarkers of inflammation in blood and feces and they share a non-resolving pattern of gut mucosal inflammation.

Both the innate and the adaptive immune system play important roles in the intestinal inflammation. T-lymphocytes comprise a complex collection of highly differentiated T-cell subsets playing key roles in the regulation and the effector phase of the immune response. Several types of innate immune cells have been shown to contribute to IBD pathogenesis. Neutrophils perpetuate intestinal inflammation through impairment of the epithelial barrier function and release of multiple inflammatory mediators [de Souza H S, and Fiocchi C, 2016]. Dendritic cells (DCs) control crosstalk between the innate and the adaptive immunity to maintain homeostasis [Bani Ahluwalia et al., 2018]. Numerous studies have investigated the potential differences in immune effector mechanisms between the two subcategories of IBD. Neutrophil response is strongly featured in UC [Wysoczanski R et al., 2019]; in CD, neutrophil function may be impaired with reduced migration, superoxide production, and phagocytic functions [Hayee B et al., 2011, Segal A W. 2019]. There is also considerable evidence that defective mucosal immunoregulation, including abnormal changes of T cells, B cells, granulocytes, macrophages and the cytokines and chemokines produced by these cells, plays a major role in UC pathogenesis [Kaur A et al., 2020]. The high diversity in therapeutic repertoire and pharmacological mechanisms indicates the heterogeneity of disease and multiple pathways involved in molecular or cell-level pathology [Bani Ahluwalia et al., 2018].

### Biomarkers in IBD

Early diagnosis and immediate, effective therapy are crucial to prevent disease progress, functional disability and worsened quality of life. Contrary to such a wealth of research, to date, there are still no ideal serum biomarkers for IBD. Serum profiling and non-coding RNAs are just starting to blossom but reveal great promise for future clinical practice. Recently, serum biomarkers have made a great progress and become a focus in IBD study because they are non-invasive, convenient, and relatively inexpensive compared to markers in biopsy tissue, stool, breath, and other body fluids [Chen P et al., 2020]. Adequate monitoring is crucial for identifying disease relapse and administering timely treatments. Treatments should be individualized and adjusted during the disease course on the basis of disease severity, extent of lesions, disease behavior, and responses to drugs. Responses to treatments must be closely monitored to determine effectiveness and avoid disease complications.

IBD biomarkers have been identified in colonic tissue, blood, stool, urine, and breath. Blood-based biomarkers are non-invasive, can be readily obtained, are not easily contaminated, and are the most widely used. An ideal biomarker should be non-invasive, sensitive, disease specific, easy to perform, and cost-effective [Vermeire S. 2006]. To date, there is no ideal biomarker that possesses all the aforementioned qualities to accurately diagnose IBD, to differentiate between subtypes of IBD, or to monitor disease activity.

### Present Treatment Options in IBD

The existing IBD treatment algorithm relies on sequential treatment of patients with successive therapeutic classes on failure of the initial therapy. This results in treating patients, often for extended periods of time, with expensive yet ineffective therapies. As one thinks of providing sustainable global IBD care, the ability to match patient and therapy more precisely is essential to avoid unnecessary drugs and provide more cost-effective care. Research has consistently shown that up-front effective treatment early on in the course of disease is more effective than an incremental step-up treatment approach [D'Haens G et al., 2008].

Current therapeutic approaches are focused on inhibiting the aberrant gut inflammation and immune response. The past few years have seen an expansion in IBD therapeutic options. The conventional approach to treat patients with IBD is to induce and maintain clinical remission, focusing on mucosal healing and preventing long-term complications in order to achieve the best attainable quality of life for patients [Bani Ahluwalia et al. 2008]. Conventional treatments control symptoms through pharmacotherapy, including aminosalicylates, corticosteroids (CSs), immunomodulators, and biologics, with other general measures and/or surgical resection if necessary. Recent progress in therapeutic approaches, especially the emergence of biologics, has not only promoted the transformation of the treatment mode in IBD, but also changed the perspective of IBD therapy. Traditionally, the therapeutic effects are mainly evaluated through clinical symptom score. Nowadays, the disease activity can also be assessed by objective indicators such as endoscopic findings and biomarkers using treat-to-target approach [Nakase H et al., 2021]. The goals are not only to induce and maintain remission in symptom, to prevent and treat complications but also to achieve mucosal and histological healing.

Despite the advanced treatment options, the rates of primary non-response, loss of response, or adverse reactions affect more than 50% of all patients indicating the unmet need for further treatment options. Small-molecule drugs (SMD) represent one of the most promising novelties in the IBD therapeutic pipeline. The main advantages of SMD over biologics include the short half-life, the lower risk of immunogenicity and the oral administration, which obviously increase the patients' compliance and quality of life.

Despite that multiple drugs are available for treatment of IBD, a large proportion of patients either have no response or lose response to therapy. Approximately 30% of CD patients fail to respond to infliximab or adalimumab (anti-TNFs), and the annual risk of losing response to infliximab is about 13% per patient year [Adegbola, S. O. et al. 2018]. For the past few years, advances in novel IBD treatments have provided new options for these patients. Gut-specific-α4β7 integrin antibody (vedolizumab and etrolizumab) and IL-12/IL-23 inhibitors (ustekinumab) have shown therapeutic effect to patients who have refractory IBD or lose response to anti-TNF treatment [Hazel K. and O'Connor A. 2020; Liefferinckx C et al. 2019]. As more and more new therapies are put in clinical practice, (like S1P inhibitors ozanimod and etrazimod) precision medicine will become a vital challenge for IBD treatment. This involves accurate stratification of patients, the use of effective and reliable biomarkers, and the determination of the optimal clinical pathways for different individuals [Chen P. et al. 2020]. Following the success of biologics in the clinical management of IBD patients, there has been intensive research for alternative effective anti-cytokine strategies. Tofacitinib (CP-690,550) is the first-in-class, oral, pan-Janus kinase (JAK) inhibitor known to be effective and safe for moderate-to-severe UC patients [Weisshof, R. et al. 2018].

### Multidrug Transporters in Health and Disease

Multidrug resistance (MDR-ABC) transporters (MDR1/P-gp/ABCB1; MRP1/ABCC1; BCRP/ABCG2) are important components in the development of drug resistance in malignancies [Gottesman, M. M. et al., 2002] and in autoimmune conditions, such as rheumatoid arthritis (RA) [Marki-Zay, J., et al., 2013]. The most important classical systemic disease-modifying anti-rheumatic drugs (csDMARDs; including methotrexate (MTX), sulfasalazine, leflunomide and hydroxychloroquine) are substrates of MDR proteins.

Regarding IBD, scientific literature in relation to multidrug transporters is less extensive than for RA. Studies mainly focus on genetic factors playing an important role in susceptibility to IBD [Petryszyn, P., 2021, Cao Y 2015] or on the role of transporter proteins in glucocorticoid resistance [De Iudicibus S, 2011, Farrell R J, 2000].

The well-known phenomenon of multidrug resistance as it is observed clinically in conventional chemotherapy or in case of conventional DMARD therapy in RA is less reported, even if the role of MDR1 is studied. Several cDMARDs frequently administered in first-line IBD therapy is known to be MDR1 substrate and emerging evidence supports that additional newly approved small molecule drugs (Jakinibs) are transported by MDR1 or BCRP [European Medicines Agency Public Assessment Reports EMA/CHMP/853224/2016, EMA/13493/2017, EMA/424374/2020, EMA/608624/2019 Corr., EMA/705612/2020, EMA/647846/2021].

Cario, Elke [Cario, Elke (2017)] has written that multidrug transporter ABCB1 (also called MDR1 or P-glycoprotein, P-gp) plays a key role in protecting the gastrointestinal barrier from xenobiotic accumulation. Altered expression of ABCB1 may contribute to the development and persistence of chronic intestinal inflammation in IBD. While decreased efflux activity of P-gp may promote disease susceptibility and drug toxicity, increased efflux activity may impair drug responses in IBD and may confer resistance to therapeutic drugs in IBD. However, the potential therapeutic value of MDR1 as a molecular target requires further clarification.

Drozdzik, Marek et al. [Drozdzik, Marek et al. (2020)] summarized that gastrointestinal (e.g. IBD) and systemic pathology states may affect expression and function of membrane transporters in the gastrointestinal tract. In case of MDR1 (ABCB1), altered levels of P-gp may affect the treatment outcome of IBD. Expression of P-gp may be involved in modulation of drug response to 5-amino salicylic acid, immunosuppressant drugs and corticosteroids. According to the evidence, patients administered with 5-aminosalicylates and not responding to the medication demonstrated a marked suppression in P-gp expression in the inflamed mucosa in comparison with the unaffected tissue. Thus, measurement was made on samples obtained from the site of inflammation. Some of glucocorticoids were defined to be substrates of P-gp, thus its altered status might affect drug response. Another MDR transporter, BCRP (also called ABCG2) may also affect response to 5-amino salicylic acid, corticosteroids and immunosuppressants. According to reports, patients treated with 5-aminosalicylates and not responding to the drug showed a considerable downregulation of BCRP in the inflamed mucosa (in comparison with the unaffected tissue). In this review article, levels of the transporters were usually expression levels (for example mRNA expression level).

Chen, Yan et al. [Chen, Yan et al. (2022)] compared the effects of aminosalicylic acid, glucocorticoids and immunosuppressants on the expression levels of multidrug resistance genes in patients with ulcerative colitis (UC), with the aim of providing a theoretical and therapeutic basis for the diagnosis, treatment, and prevention of UC. They quantified the mRNA expression levels of MDR1 gene before and after a given treatment. According to their results, administration of 5-aminosalicylic acid (5-ASA) drugs did not correlate with MDR1 expression in UC, whereas administration of glucocorticoids and immunosuppressive drugs was positively correlated with MDR1 expression profile. The expression levels of MDR1 mRNA and its product P-gp were significantly upregulated in patients who did not respond to glucocorticoids and immunosuppressive drugs. 5-ASA had no effect on the expression levels of MDR1 and its product P-gp in patients with a confirmed diagnosis of UC. It is important to note here, that this result is contradictory to the findings of Drozdzik et al. (2020). Chen et al. also found that the use of glucocorticoids and immunosuppressants can increase the expression level of MDR1. The RNA expression was measured in colonic mucosal tissue specimens, endoscopic pathological mucosal tissues or postoperative pathological biopsies, i.e. samples obtained directly from the sites of inflammation.

Sales-Campos, H. et al. [Sales-Campos, H. et al. (2015)] teach that the classical therapeutic strategies aim to control the exacerbated host immune response with aminosalicylates, antibiotics, corticosteroids, thiopurines, methotrexate, i.e. classic immunosuppressive agent and antitumor necrosis factor (TNF) biological agents. Although successful in the treatment of several CD or UC conditions, these drugs have limited effectiveness, and variable responses may culminate in unpredictable outcomes. The authors suggest that an ideal therapy should reduce inflammation without inducing immunosuppression, however, admit, that this remains a challenge.

Zenlea T, et al. [Zenlea T, et al. 2014] review the mechanism of action, clinical efficacy and adverse effects of “conventional” immunomodulators including azathioprine, 6-mercaptopurine, methotrexate, and cyclosporine and biologics including anti-tumor necrosis factor (TNF) agents as well as adhesion molecule inhibitors and suggest that while the underlying genetic and molecular pathways responsible for the development and severity of IBD are still poorly understood, and the therapeutic focus has been on immunosuppressive medications, the goal of therapy still remains the maintenance of a steroid-free remission.

WO2017/223409A1 discloses methods and compositions for monitoring MDR1 transporter function in human IBD patients; as well as methods for stratification of IBD patients for bile acid directed therapies, and therapeutic methods that integrate the patient stratification with bile acid sequestration therapies. In a method, MDR1 activity is quantified in human T cells in a biological sample. The T cells are selected from CD4⁺ effector/memory, CD8⁺ naïve, CD8⁺ effector/memory and CD8⁺ short-lived effector/memory T cells. Preferably, the MDR1 function is assayed via determining the frequency of MDR1⁺ cells in T cell subsets. In another method, a human patient with IBD is identified for treatment with bile acid sequestration therapies, wherein a substantive decrease of the combined MDR1+ cell frequency of the candidate IBD patient compared to that of a healthy control subject identifies the candidate IBD patient as one suitable for bile acid sequestration therapies. In short, detecting MDR1 loss-of-function in a patient indicates the suitability for said therapies.

CN104535774 discloses a new detection index for drug sensitivity under the state of an inflammatory bowel disease and application of the new detection index in the design of a drug therapy scheme. If expression levels of P-gp is elevated in peripheral blood lymphocyte, drug resistance may occur. However, P-gp specific inhibitors can reverse this drug resistance. Furthermore, P-gp expression levels of peripheral blood lymphocyte can be used for evaluating the drug resistance of an IBD patient.

US2009258848A1 discloses methods and kits for determining inflammatory bowel disease status in a subject, as well as methods of identifying biomarker for determining IBD status in a subject. According to the invention, about 400 biomarkers are suitable for determining IBD status—for example ABCB1 gene, whose expression is downregulated in IBD patients.

Despite a wealth of research in the subject, there is still the need to provide appropriate biomarkers to accurately diagnose IBD, to differentiate between subtypes of IBD, or to monitor disease activity. Many unanswered questions remain regarding the role of MDR transporters in the diagnosis of IBD and whether they are suitable as biomarkers for predicting the effectiveness or success of a therapy for IBD patients.

The present inventors' method is useful to provide in vitro diagnostic methods for predicting non-responsiveness of IBD patients to immunosuppressive agents, as well as in vitro methods for assessing the status of an IBD patient previously treated with an immunosuppressive agent and thereby support or carry out IBD therapy.

### BRIEF DESCRIPTION OF THE INVENTION

1. The invention relates to an in vitro method for predicting the responsiveness of an IBD patient to a therapy with an intracellularly acting immunosuppressive agent of interest, said method comprising the steps of
   providing a sample of said IBD patient from a biological sample taken from said IBD patient at an initial period of a treatment with the immunosuppressive agent of interest, preferably a time before treatment with the immunosuppressive agent of interest or up to the 4^{th} month of treatment with the immunosuppressive agent of interest,
      said sample comprising effector mononuclear cells (preferably lymphocytes and/or monocytes and/or NK cells) from said patient,
   obtaining a transporter activity level by measuring transport activity by a multidrug ABC transporter (preferably selected from the group consisting of B, C and G class of ABC transporters, more preferably the group consisting of B and C class, or the group consisting of B and G class), in the effector mononuclear cells in said sample,
   comparing the transporter activity level with a reference transporter activity level,
   considering said patient as a non-responder to the therapy with the intracellularly acting immunosuppressive agent of interest when the transporter activity level is significantly different from the respective reference transporter activity level.
The invention also relates to an in vitro method for predicting the responsiveness of an IBD patient to a therapy with an intracellularly acting immunosuppressive agent of interest, said method comprising the steps of
   providing a sample of said IBD patient from a biological sample taken from said IBD patient at a time before treatment with the immunosuppressive agent of interest or up to the 4^{th} month of treatment with the immunosuppressive agent of interest,
      said sample comprising effector mononuclear cells (preferably lymphocytes and/or monocytes and/or NK cells) from said patient,
   obtaining one or more transporter activity value(s) by measuring transport activity by one or more multidrug ABC transporter(s) selected from the group consisting of B, C and G class of ABC transporters (preferably the group consisting of B and C class, or the group consisting of B and G class), in the effector mononuclear cells in said sample,
   comparing the one or more transporter activity value(s) with one or more reference transporter activity value(s),
   considering said patient as a non-responder to the therapy with the intracellularly acting immunosuppressive agent of interest when the one or more or each transporter activity value(s)) is/are significantly different from the respective reference transporter activity (value(s).
Preferably the method is a diagnostic method.
Preferably the method is a diagnostic method combined with a treatment with the intracellularly acting immunosuppressive agent of interest. Preferably the method is a method for treatment with the intracellularly acting immunosuppressive agent of interest provided that the patient is responsive to said agent. Preferably the method also comprises a treatment with the intracellularly acting immunosuppressive agent of interest, provided that the patient has not been found non-responsive to said agent.
In a preferred embodiment the invention relates to the in vitro method for predicting the responsiveness, wherein at the time of taking a sample
the patient is treated with the intracellularly acting immunosuppressive agent of interest, (and/)or.
the patient is naïve for the intracellularly acting immunosuppressive agent of interest.
Preferably, the intracellularly acting immunosuppressive agent of interest is a conventional immunosuppressive agent. Preferably, the intracellularly acting immunosuppressive agent of interest is a classic immunosuppressive agent.
In a preferred embodiment the invention relates to the in vitro diagnostic method for predicting the responsiveness, wherein
   multiple samples of said IBD patient are provided each sample from biological samples taken from said IBD patient at multiple points of time, and
   said one or more transporter activity value(s) is/are obtained from each sample,
   comparing the transporter activity value(s) from each sample with one or more reference transporter activity values for the same point of time in which the respective biological sample is taken,
   considering said patient as a non-responder to the therapy with the intracellularly acting immunosuppressive agent of interest when the level indicated by said one or more transporter activity value(s) for at least one, preferably for each sample is significantly different from the respective level indicated by one or more reference transporter activity value(s).
2. The invention also relates to an in vitro diagnostic method of paragraph 1 for assessing the status of a IBD patient treated with a previous intracellularly acting immunosuppressive agent, to predict whether said patient is non-responsive to the intracellularly acting immunosuppressive agent of interest, said method comprising the steps of
   providing a biological sample of said IBD patient, said sample comprising effector mononuclear cells (preferably lymphocytes and/or monocytes and/or NK cells) from said patient,
   obtaining a transporter activity level (or one or more transporter activity value(s)) by measuring transport activity of a multidrug ABC transporter (or of one or more multidrug ABC transporter(s)) selected from the group consisting of B, C, or G class of ABC transporters, in the effector mononuclear cells in said sample,
   comparing the transporter activity level (or one or more transporter activity value(s)) with a reference transporter activity level (or one or more reference transporter activity values),
   considering said patient as a non-responder to the therapy with the intracellularly acting immunosuppressive agent of interest when the transporter activity level (or one or more or each transporter activity value) is significantly different from the respective reference transporter activity level (or one or more reference transporter activity value(s)).
3. Preferably the invention also relates to the in vitro method according to any of paragraphs 1 to 2, wherein the biological sample is blood-derived sample.
4. Preferably the invention also relates to the in vitro method according to paragraph 3, wherein the effector mononuclear cells are selected from the group consisting of lymphocytes, monocytes and NK cells, preferably CD3+T-lymphocytes, CD14+ monocytes, CD16+NK cells or neutrophil granulocytes.
5. The in vitro method according to any of paragraphs 1 to 4, wherein the intracellularly acting immunosuppressive is a small molecule drug used in IBD, said small molecule drug being selected from
   a glucocorticoid, optionally a glucocorticoid combined with conventional disease modifying drug, e.g. a DMARD (cDMARD),
   a conventional disease modifying drug, e.g DMARD (cDMARD) without a glucocorticoid,
   a steroid,
   a tsDMARD, and
wherein the ABC transporters are selected from the group consisting of B and G class preferably of ABCB1 (MDR1), ABCC1 (MRP1) and ABCG2 (BCRP).
In an embodiment the steroid is a systemic steroid, preferably selected from the group consisting of prednisolone, methylprednisolone, prednisone, hydrocortisone.
6. Preferably the invention also relates to the in vitro method according to any of paragraphs 2 to 5, wherein the previous intracellularly acting immunosuppressive agent is a small molecule drug used in IBD, said small molecule drug being an anti-inflammatory agent, preferably a steroid anti-inflammatory agent or a non-steroid anti-inflammatory agent (NSAID). Preferably the steroid is a systemic steroid, preferably selected from the group consisting of prednisolone, methylprednisolone, prednisone, hydrocortisone.
7. Preferably the invention also relates to the in vitro method according to any of paragraphs 2 to 4, wherein the previous intracellularly acting immunosuppressive is a small molecule drug used in IBD, said small molecule drug being
a glucocorticoid, optionally a glucocorticoid combined with conventional disease modifying drug, e.g. a DMARD (cDMARD), or
a conventional disease modifying drug, e.g DMARD (cDMARD) without a glucocorticoid.
8. Preferably the invention also relates to the in vitro diagnostic method according to any of paragraphs 1 to 7, preferably to paragraph 6 or 7, wherein the intracellularly acting immunosuppressive agent of interest is a cDMARD.
9. Preferably the invention also relates to the in vitro method according to any of paragraphs 1 to 8, preferably any of paragraphs 2 to 4 and 6 to 8, wherein the ABC transporters are selected from the group consisting of B and G class preferably of ABCB1 (MDR1) and ABCG2 (BCRP).
10. Preferably the invention also relates to the in vitro method according to paragraph 7, wherein the intracellularly acting immunosuppressive agent of interest is different from a steroid and is different from a NSAID.
Preferably the second intracellularly acting immunosuppressive is a small molecule anti-IBD drug.
Preferably the second intracellularly acting immunosuppressive is a tsDMARD, preferably a selective or non-selective (pan) Janus-kinase (JAK) inhibitor.
11. Preferably the invention also relates to the in vitro method according to any of paragraphs 1 to 10, or any of paragraphs 2 to 4 and 6 to 10, wherein the patient is or before starting the treatment with it, has been naïve for the intracellularly acting immunosuppressive agent of interest.
12. The in vitro diagnostic method according to paragraph 1 to 7 or paragraph 9 to 11, wherein the intracellularly acting immunosuppressive agent of interest is a tsDMARD, preferably said tsDMARD being selected from a JAK inhibitors, S1PR agonist and a PDE4 inhibitor, preferably a JAK inhibitor, wherein preferably
13. Preferably the invention also relates to the in vitro diagnostic method according to any of paragraphs 1 to 7 or paragraphs 10 to 12, wherein the ABC transporters are selected from the group consisting of B and C class of ABC transporters, preferably of ABCB1 (MDR1) and ABCC1 (MRP1).
14. Preferably the invention also relates to the in vitro method according to any of paragraphs 1 to 14 preferably any of paragraphs 1 to 10 and 12 to 14 wherein the patient having been treated for a given period of time by the intracellularly acting immunosuppressive agent of interest, preferably said period being selected from the group consisting of at least 2 weeks, 4 weeks, 1 month, 2 months and 3 months.
15. Preferably the invention also relates to the in vitro method according to paragraph 1 to 14 wherein said method is a diagnostic method. Preferably the method is a diagnostic method combined with a treatment with the intracellularly acting immunosuppressive agent of interest. Preferably the method is a method for treatment with the intracellularly acting immunosuppressive agent of interest provided that the patient is responsive to said agent. Preferably the method also comprises a treatment with the intracellularly acting immunosuppressive agent of interest, provided that the patient has not been found non-responsive to said agent.
16. Preferably the invention also relates to the in vitro diagnostic method of any of paragraphs 10 to 15, wherein the cells are lymphocytes, preferably CD3+/CD8+ lymphocytes and MDR1 transport activity is measured in said sample, to obtain MDR1 transport activity value(s)
   comparing said MDR1 transport activity value(s) with one or more reference values
      said reference value(s) being or having been obtained from patients having a low inflammatory status,
considering said patient as a non-responder to the said JAK inhibitor therapy if the MDR1 transport activity values obtained from said sample are significantly lower than said one or more reference values.
Preferably the cells are lymphocytes, preferably CD3⁺/CD8⁺ lymphocytes and MDR1 transport activity is measured in said sample, to obtain MDR1 transport activity value(s)
   comparing said MDR1 transport activity value(s) with one or more reference values
      said reference value(s) being or having been obtained from patients who are naïve for said treatment,
   considering said patient as a non-responder to the said JAK inhibitor therapy if the MDR1 transport activity values obtained from said sample are significantly lower than said one or more reference values
17. Preferably the invention also relates to the in vitro diagnostic method of any of paragraphs 10 to 15, wherein the cells are monocytes, preferably CD14⁺ monocytes and MDR1 transport activity is measured in said sample, to obtain MDR1 transport activity value(s)
   comparing said MDR1 transport activity value(s) with one or more reference values
      said reference value(s) being or having been obtained from patients having a low inflammatory status,
   considering said patient as a non-responder to the said JAK inhibitor therapy if the MDR1 transport activity values obtained from said sample are significantly higher than said one or more reference values.
18. Preferably the invention also relates to the in vitro diagnostic method of any of paragraphs 10 to 15, wherein the cells are monocytes, preferably CD14+ monocytes and MRP1 transport activity is measured in said sample, to obtain MRP1 transport activity value(s)
   comparing said MRP1 transport activity value(s) with one or more reference values
      said reference value(s) being or having been obtained from patients having a low inflammatory status,
   considering said patient as a non-responder to the said JAK inhibitor therapy if the MRP1 transport activity values obtained from said sample are significantly higher than said one or more reference values.
Preferably the cells are monocytes, preferably CD14⁺ monocytes and MRP1 transport activity is measured in said sample, to obtain MRP1 transport activity value(s)
   comparing said MDR1 transport activity value(s) with one or more reference values
      said reference value(s) being or having been obtained from patients who are naïve for said treatment,
   considering said patient as a non-responder to the said JAK inhibitor therapy if the MRP1 transport activity values obtained from said sample are significantly higher than said one or more reference values.
19. Preferably the invention also relates to the in vitro method of any of paragraphs 15 to 18, wherein said JAK inhibitor is tofacitinib.
20. Preferably the invention also relates to a method wherein the in vitro method of any of paragraphs 15 to 19 is carried out wherein
said IBD patient is responder to a different JAK inhibitor, and/or
a method of treatment of said IBD patient with a different JAK inhibitor is proposed and/or carried out.
Preferably in the previous claims the JAK inhibitor is a JAK1/JAK3 inhibitor.
21. The invention also relates to a method wherein the in vitro method of any of paragraphs 15 to 19 is carried out wherein
said IBD patient is non-responder to said tsDMARD and wherein a therapy (treatment) of said patient with a different anti-IBD agent, e.g.
another tsDMARD is proposed and/or carried out, and/or
a bDMARD therapy is proposed or carried out.
22. Preferably the invention also relates to the in vitro method of any of claims 1 to 20, wherein IBD is UC.
Preferably the invention also relates to the in vitro method of any of claims 1 to 21, or any of claims 2 to 4 and 6 to 9, wherein the patient is or before starting the treatment with it, has been naïve for the intracellularly acting immunosuppressive agent of interest.
23. Preferably the invention also relates to the in vitro method of any of paragraphs 5 to 9, wherein
the IBD patients are patients treated with steroid as the intracellularly acting immunosuppressive agent of interest, the cells are lymphocytes, preferably CD3+ lymphocytes and
   composite MDR1+MRP1 transport activity is measured in said sample, to obtain composite MDR1+MRP1 transport activity values
   comparing said composite MDR1+MRP1 transport activity values with one or more reference values
      said reference values being or having been obtained from patients having a low inflammatory status,
   considering said patient as a non-responder to the said steroid therapy if the composite MDR1+MRP1 transport activity values obtained from said sample are significantly higher than said one or more reference values,
preferably higher than a reference value obtained for responders of the same therapy, wherein preferably transport activity is measured after at least 2 weeks, one month or at 1 to 3 months from the starting of steroid treatment wherein optionally a reference value is a value measured at week 0.
24. Preferably the invention also relates to the in vitro method of any of paragraphs 5 to 9, wherein
the IBD patients are patients treated with a small molecule anti-IBD drug as the intracellularly acting immunosuppressive agent of interest,
the cells are lymphocytes, preferably CD3+ lymphocytes and composite MDR1+MRP1 transport activity is measured in said sample, to obtain composite MDR1+MRP1 transport activity values
   comparing said composite MDR1+MRP1 transport activity values with one or more reference values
      said reference values being or having been obtained from patients having a low inflammatory status,
   considering said patient as a non-responder to the said steroid therapy if the composite MDR1+MRP1 transport activity values obtained from said sample are significantly higher than said one or more reference values.
In a preferred embodiment a value is obtained for responders of the same therapy,
preferably transport activity is measured after at least 2 weeks, one month or at 1 to 3 months from the starting of steroid treatment wherein optionally a reference value is a value measured at week 0,
preferably wherein the small molecule anti-IBD drug is selected from steroids, azathioprine and 5-ASA.
25. Preferably the invention also relates to the in vitro method of any of paragraphs 5 to 9, wherein the IBD patients are patients treated with steroid as the intracellularly acting immunosuppressive agent of interest, the cells are lymphocytes, preferably CD3⁺ lymphocytes and
   MDR1 transport activity is measured in said sample, to obtain MDR1 transport activity value
   comparing said MDR1 transport activity values with one or more reference values
      said reference values being or having been obtained from patients having a low inflammatory status,
   considering said patient as a non-responder to the said steroid therapy if the MDR1 transport activity values obtained from said sample are significantly lower than said one or more reference values,
preferably lower than a reference value obtained for responders of the same therapy wherein preferably transport activity is measured at week 0, and/or
   considering said patient as a non-responder to the said steroid therapy if the BCRP transport activity values obtained from said sample are preferably higher than a reference value obtained for responders of the same therapy, wherein transport activity is measured at 1 to 3 months from the starting of steroid treatment wherein optionally a reference value is a value measured at the same time.
26. Preferably the invention also relates to the in vitro method of any of paragraphs 5 to 9, wherein
   MDR1 transport activity is measured in said sample, to obtain MDR1 transport activity value
   comparing said MDR1 transport activity values with one or more reference values
      said reference values being or having been obtained from patients having a low inflammatory status,
   considering said patient as a non-responder to the said steroid therapy if the MDR1 transport activity values obtained from said sample are significantly higher than said one or more reference values,
preferably higher than a value obtained for responders of the same therapy, wherein transport activity is measured after at least 2 weeks, one month or at 1 to 3 months from the starting of steroid treatment wherein optionally a reference value is a value measured at the same time or at week 0;
preferably wherein the small molecule anti-IBD drug is selected from steroids, azathioprine and 5-ASA.
27. Preferably the invention also relates to the in vitro method of any of paragraphs 5 to 9, wherein
the IBD patients are patients treated with steroid as the intracellularly acting immunosuppressive agent of interest, the cells are lymphocytes, preferably CD3⁺ lymphocytes and
   BCRP transport activity is measured in said sample, to obtain BCRP transport activity value
   comparing said BCRP transport activity values with one or more reference values
      said reference values being or having been obtained from patients having a low inflammatory status,
   considering said patient as a non-responder to the said steroid therapy if the BCRP transport activity values obtained from said sample are significantly lower or higher than said one or more reference values,
preferably lower than a reference value obtained for responders of the same therapy wherein preferably transport activity is measured at week 0, and/or
preferably
   considering said patient as a responder to said steroid therapy if the BCRP transport activity values obtained from said sample are preferably lower than a reference value obtained for non-responders of the same therapy wherein preferably transport activity is measured at week 0, and/or
   considering said patient as a non-responder to the said steroid therapy if the BCRP transport activity values obtained from said sample are preferably higher than a reference value obtained for responders of the same therapy, wherein transport activity is measured at 1 to 3 months from the starting of steroid treatment
wherein optionally a reference value is a value measured at the same time.
28. Preferably the invention also relates to the in vitro diagnostic method of any of claims 5 to 9, wherein
the IBD patients are patients treated with small molecule anti-IBD drug as the intracellularly acting immunosuppressive agent of interest,
   BCRP transport activity is measured in said sample, to obtain BCRP transport activity value
   comparing said BCRP transport activity values with one or more reference values
      said reference values being or having been obtained from patients having a low inflammatory status,
   considering said patient as a non-responder to the said small molecule anti-IBD drug therapy if the BCRP transport activity values obtained from said sample are significantly lower or higher than said one or more reference values, preferably lower than a reference value obtained for responders of the same therapy wherein preferably transport activity is measured at week 0, and/or
preferably
   considering said patient as a responder to said small molecule anti-IBD drug therapy if the BCRP transport activity values obtained from said sample are preferably lower than a reference value obtained for non-responders of the same therapy wherein preferably transport activity is measured at week 0, and/or
   considering said patient as a non-responder to said small molecule anti-IBD drug therapy if the BCRP transport activity values obtained from said sample are preferably higher than a reference value obtained for responders of the same therapy, wherein transport activity is measured at 1 to 3 months from the starting of said small molecule treatment
   wherein optionally a reference value is a value measured at the same time.
Preferably the small molecule anti-IBD drug is selected from steroids, azathioprine and 5-ASA.
The invention also relates to a method wherein the method of any of paragraphs 23 to 28 is carried out wherein said IBD patient is non-responder to said small molecule anti-IBD agent, wherein preferably said anti-IBD agent is selected from steroids, azathioprine and 5-ASA, and wherein therapy (treatment) of said patient
with another small molecule anti-IBD agent is proposed and/or carried out, and/or
with a cDMARD of csDMARD is proposed or carried out.
29. Preferably the invention also relates to the in vitro diagnostic method of any of paragraphs 1 to 28 wherein one or more reference transporter activity values is/are selected from
   comparing the one or more transporter activity value(s) with one or more pre-determined threshold transporter activity level(s), one or more pre-determined threshold transporter activity level(s),
wherein each pre-determined threshold transporter activity level is a threshold value for the transport activity of said one or more multidrug transporters and which has been determined using the same one or more substrates.
30. Preferably said threshold transporter activity level is or has been determined by using the one or more substrates by
measuring or quantifying transport activity of said one or more multidrug transporters in the said effector mononuclear cells in a (reference) patient group known to be responder to the therapy and a (reference) patient group known to be non-responder to the therapy, and
the transport activity values measured in the responder and non-responder patient groups are analysed (preferably statistically analysed as distributions) to find a threshold level which differentiates between responder transport activity values and non-responder transport activity values.
31. In a highly preferred embodiment the substrate is a fluorescent substrate.
In a highly preferred embodiment the substrate is a compound which is converted by intracellular enzymes into a form having a different transport property and/or a different fluorescent spectrum. Preferably the compound is an ester hydrolyzed by esterases. In a preferred embodiment the substrate is a calcein ester, preferably calcein AM.
In a preferred embodiment the substrate is a fluorescein and/or rhodamine derivative ester compound, preferably comprising a phenantrene derivative moiety.
In a highly preferred embodiment the substrate is a fluorescein derivative ester compound is a PhenGreen compound
In a highly preferred embodiment the activity is quantified as a multidrug activity factor (MAF).
32. Preferably the patient needs a switch or modification of the cDMARD therapy and switching to tsDMARD therapy due to documented cDMARD therapy failure or drug intolerance.
33. In a preferred embodiment the method is a treatment method wherein an IBD patient is treated by an intracellularly acting immunosuppressive agent of interest, provided that said patient is
not found non-responsive by the method of the invention to said agent of interest, or
is found responsive by the method of the invention to said agent of interest.
34. In a preferred alternative variant, the invention relates to an in vitro diagnostic method for monitoring the status of IBD in a patient under therapy by an intracellularly acting immunosuppressive agent of interest, said method comprising the steps of
   providing a biological sample of said IBD patient, said sample comprising effector mononuclear cells (preferably lymphocytes and/or monocytes and/or NK cells) from said patient,
   obtaining an initial transporter activity level (one or more transporter activity value(s)) by measuring transport activity of a multidrug ABC transporter (of one or more multidrug ABC transporter(s)) selected from the group consisting of B, C, or G class of ABC transporters, in the effector mononuclear cells in said sample, (to obtain one or more transporter activity value(s)),
   obtaining a follow-up transporter activity level (one or more transporter activity value(s)) by measuring transport activity of a multidrug ABC transporter (of one or more multidrug ABC transporter(s)) selected from the group consisting of B, C, or G class of ABC transporters, in the effector mononuclear cells in said sample, (to obtain one or more transporter activity value(s)),
   comparing the follow-up transporter activity level (or one or more transporter activity value(s)) with an initial transporter activity level (or one or more reference transporter activity values),
   assessing the progress or amelioration of the condition of the patient when the follow-up transporter activity level (or one or more or each transporter activity value) is significantly different from the initial transporter activity level (or one or more reference transporter activity value(s)).

### Definitions

“Measuring” or “measurement” is understood herein as quantitative characterization of a physical object or entity or a multitude (population or plurality) thereof, or their function or quantitative characterization of a physical or chemical process, comprising the assignment of a quantity, value, e.g. a numerical value or a number characteristic of the object or entity or multitude or function or process, by comparison with units and, in comparison with other object or entity or multitude or function or process. Preferably a measurement is consistent with methods known in the art or the international guidelines of metrology. The magnitude is the numerical value of the characterization, usually obtained with a suitably chosen measuring instrument, whereas the unit assigns a mathematical weighting factor to the magnitude that is derived as a ratio to the property of an artefact used as standard unit or a natural physical quantity as unit.

“Quantifying” or “quantification” or “quantitation” is understood herein as an assignment of a physical quantity to a physical object or entity or a multitude (population or plurality) thereof, or their function or quantitative characterization of a physical or chemical process, expressed in a numerical value or number and units, and, in comparison with other object or entity. Preferably “quantifying” or “quantification” is a measurement or an essential part of a measurement.

The measurement has an uncertainty which may represent the random and systemic errors of the measurement procedure. The skilled person is aware of this and can handle this error in view of the measurement or quantification applied.

“Comparing” two levels preferably two activity levels are understood herein to include a comparison of quantities expressed in numerical values characterizing said levels to establish which is higher or lower, or establishing a difference or establishing a ratio of the levels, or values derived from the levels, optionally completed with other mathematical procedures as the quantification or calculation method requires.

A “membrane transporter” is a membrane integrated protein, which is permanently anchored in the membrane having a membrane spanning part and having parts on both sides of the membrane, wherein it is capable of transporting, e.g. exporting or extruding or importing entities, either actively or passively through the membrane into which it is integrated in. The entity can be e.g. a molecule or a molecule ion which is preferably fluorescent.

“ABC transporter” stands for ATP-binding cassette transporters which are a superfamily of membrane transporters that utilize the energy of adenosine triphosphate (ATP) hydrolysis to carry out certain biological processes including transport of entities across membranes. Denominations and subfamilies of ABC transporters are used herein as assigned by the HUGO Gene Nomenclature Committee (HGNC). For example, membrane transporters of the “ABCG family” belong to the G subfamily of ABC transporters consisting of half-transporters, which oligomerise to form the functional transporter.

A “multidrug transporter” is an ABC transporter, also mentioned herein as an ABC multidrug transporter, which can transport from the cell, in the membrane of which it is present, a multiplicity or preferably a wide variety of chemical compounds.

“Multidrug resistance”, as used herein, refers to the ability of cells to develop resistance to a broad range of structurally or functionally unrelated drugs by multidrug transporter(s). Preferably, “multi-drug resistance” refers to the state which is dependent on expression or overexpression of MDR1, MRP1, or a related homologue, and/or on amplification of a gene encoding said multi-drug transporter protein.

“ABC transporter activity”, i.e. the “activity” of an ABC transporter protein refers to any activity exerted by the said transporter protein including e.g. its biological function, “transport activity”, i.e. transport of a drug through the membrane carrying the said protein, or ATP-ase activity, as far as it is an indicator of transport activity, like substrate stimulated ATP-ase activity. Preferably the activity measured in the present invention characterized or is related to or correlates to transport activity of the multidrug transporter.

A “substrate” of an ABC transporter protein is a compound that can be transported from the cell through an ABC transporter mediated active transport mechanism.

In a preferred embodiment the ABC multidrug transporters the activity of which is measured in the present invention are selected from the following transporters:
ABCB1 (MDR1) which belongs to the “ABCB family” belong to the B subfamily of ABC transporters;
ABCC1 (MRP1) which belongs to Multidrug Resistance Proteins (MRPs) of the “ABCC family” of the C subfamily of ABC transporters;
and in a preferred embodiment ABCG2 (other names among others: BRCP, MXR1, CDw338) which belong to membrane transporters of the “ABCG family” i.e. of the G subfamily of ABC transporters consisting of half-transporters, which oligomerise to form the functional transporter.

By “measuring transport activity” of a multidrug transporter it is understood that in cells or in a population of cells in which the given multidrug transporter resides or assumably resides the transport activity is measured and/or quantified wherein preferably the activity is total or overall transport activity of one or more multidrug transporter(s). In a preferred embodiment if the expression of the transporters is increased the activity also increases.

In general, any physical quantity which quantitatively characterizes the transport activity of the multidrug transporter can be applied in the present invention. In a preferred embodiment the physical quantity is obtained by comparison of a value obtained for cells in which the multidrug transporter is active with a value obtained for cells in which it is inhibited.

In a preferred embodiment the activity is measured via or with a substrate compound which is able to get through the cell membrane and to be transported from the cell in which the transporter resides.

In a particular preferred embodiment the activity is measured via or with a substrate compound which is a “derivative of a detectable fluorescent compound” and is able to get through the cell membrane and to be transported from the cell in which the transporter resides, wherein within the cell the derivative is converted to the detectable fluorescent compound which is non-membrane permeable, which is preferably hydrophilic or charged, and which is preferably not a substrate of the transporter protein.

Preferably the derivative is an ester derivative.

In a preferred embodiment the substrate is a fluorescein and/or rhodamine derivative ester compound, preferably comprising a phenantrene derivative moiety.

Preferably the detectable fluorescent compound is detected within the cell.

Upon measuring or quantifying (or any equivalent expression) transport activity in a preferred embodiment the activity is quantitatively characterized by a factor which comprises or is related or is proportional to the difference between the fluorescence of the detectable fluorescent compound in the cells measured in the presence of an inhibitor of the one or more membrane transporter and the fluorescence measured in absence of said inhibitor i.e. when the transporter is active. This difference is higher if the transporter is active because the fluorescence provide by the compound in the cell in lack of the inhibitor is lower as the compound (or in case its derivative is applied the derivative) is transported.

By “measuring the derivative of a detectable fluorescent compound” or the fluorescent compound is meant determining the amount of the compound which accumulates in a cell as an inverse indication of the amount of compound extruded from the cell by a multi-drug transporter protein.

Techniques for measuring intracellular calcein include, but are not limited to, flow cytometry, fluorimetry, or cell imaging.

In a preferred embodiment the transport activity of the MDR transporter is measured via the transport (extrusion) of the derivative compound as the difference between the amount of the dye accumulated in the presence and absence of inhibitors. The fluorescence measurement in the presence of an inhibitor constitutes the maximal (potential) fluorescence (Fmax) with the given cell population when the multidrug transporters are rendered non-functional. The fluorescence measurement in the absence of an inhibitor constitutes the minimal fluorescence (F₀) with the given cell population when the multidrug transporters are functional. This represents a standardization method, which eliminates unknown cell type-specific variables that influence cellular calcein accumulation, such as esterase activity, cell size, etc.

In a preferred embodiment quantitation or quantifying of this fluorescence is carried out through the development of the MDR Activity Factor (MAF) which is calculated as the ratio of the said difference (Fmax−F₀) and of the maximal fluorescence, i.e.

$$MAF = ( {F}_{max} - {F}_{o} ) / {F}_{max}$$

or if expressed in percentage,

$$MAF = 100 \times ( {F}_{max} - {F}_{o} ) / {F}_{max} .$$

MAF in percentage is often given as MAF %, however, in the present description this is not indicated, however, the MAF values given herein are given in percentage unless otherwise indicated.

The transport activity of multidrug transporters, if the compound is substrate for each of them, can be easily distinguished with selective inhibitors. Optionally other membrane transporters can be inhibited.

A “transporter activity level as used herein is a quantitative (i.e. quantified) result of the measurement of a transporter activity as defined herein. The transporter activity level is a physical quantity characterizing transporter activity, preferably substrate transport activity preferably in a sample. Preferably a transporter activity level characterizes (is the measure of) transporter activity in a biological sample, preferably taken from a subject or patient and/or characterizes transporter activity in a subject. Preferably a transporter activity level relates to substrate transport activity, e.g, wherein is obtained by way of direct or indirect measurement of substrate transport. A “transporter activity level” may be expressed in the form of a (i.e. any) mathematical and/or physical value (“transporter activity value”) suitable to characterize the transporter activity, e.g. in MAF value or other known ways characterizing the transporter activity. The “transporter activity level” or “transporter activity value” may be expressed in the form of a single value or a set of values (“transporter activity value(s)”), e.g. a distribution (e.g. statistical distribution) of values or a range of values or as a threshold value (meaning optionally a range of values lower or higher than the threshold value including or excluding the threshold value). It may also involve a range of values or a value plus a standard error e.g. with a confidence interval.

A reference transporter activity level (optionally expressed as value(s)) as used herein is a “transporter activity level” characterizing the transporter activity in a patient or in patients, e.g. in a group of subjects or patients, which serves as a reference (i.e. for comparison) for the IBD patient(s) e.g. a group of patients of interest, i.e. in whom as assessment, i.e. prediction herein, about responsiveness to the therapy with the intracellularly acting immunosuppressive agent of interest is to be made. The reference transporter activity value may characterize responsive patient(s) or group of patients; in an embodiment the reference transporter activity value may characterize a healthy subject (patient) or group of subjects (patients); in an embodiment the reference transporter activity value may characterize a drug-naïve patient(s) or group of patients. In an embodiment multiple reference activity values are applied and the measured transporter activity value is compared with multiple reference activity values and prediction of responsibility is made based on the multiple comparisons.

The reference transporter activity level (value), expressed as a mathematical and/or physical quantity, should be comparable with the transporter activity value (level) measured in the method of the invention, for example, is obtained for the same transporter. Thus, if, for example, transporter activity value in the method of the invention is a MAF value (indicative of the transporter activity level e.g. in a subject or patient), the reference transporter activity value is a MAF value (indicating the transporter activity level in a reference subject or group of subjects) is obtained for the same transporter. A transporter may indicate multiple transporters either as a source of multiple activity values or a composite activity value.

If applicable, the reference transporter activity value is determined by the same method as that used in the present invention but in a patient or in patients, e.g. in a group of patient, which serves as a reference (i.e. for comparison); optionally a mathematical (e.g. statistical) method is also applied to obtain the reference transporter activity value from multiple measurements, preferably from multiple patients to obtain a reliable reference transporter activity value. In a particular embodiment, the reference transporter activity value is obtained by
providing a biological sample of said patient(s), e.g. in a group of patient, which serves as a reference (i.e. for comparison) said sample comprising effector mononuclear cells (preferably lymphocytes and/or monocytes and/or NK cells) from said patient, and
obtaining one or more transporter activity value(s) by measuring transport activity by one or more multidrug ABC transporter(s) selected from the group consisting of B, C and G class of ABC transporters (preferably the group consisting of B and C class, or the group consisting of B and G class), in the effector mononuclear cells in said sample, wherein said transporter is the same as the transport the activity is measured in the method of the invention.

In certain embodiments the reference transporter activity value may involve a range of values or a value plus a standard error e.g. with a confidence interval. In certain embodiments the reference transporter activity value may be a threshold transporter activity value (level).

In an embodiment comparing the one or more transporter activity value(s) with one or more reference transporter activity values and/or considering said patient as a non-responder to the therapy with the intracellularly acting immunosuppressive agent of interest when the level one or more or each transporter activity value is significantly different from the respective one or more reference transporter activity value(s) involves mathematical (e.g. statistical) method(s) resulting in a consideration with a given probability and/or considering a significant difference in view of a given mathematical (e.g. statistical) method applied. Such mathematical (e.g. statistical) methods useful in diagnosis are well known for a person skilled in the art who is able to select the appropriate method providing a reliable result in the field given.

In an embodiment a “level” is or is expressed in the form of an intensive quantity.

“Comparing” two levels (e.g. in the form of a value or values) is understood herein to include a comparison to establish which is higher or lower, or establishing a difference or establishing a ratio of the levels, or values derived from the levels, optionally completed with other mathematical procedures (calculation) as the measurement method requires. In an embodiment comparing comprises subtracting two levels, e.g. subtracting two normalized or baseline-corrected level. In an embodiment comparing comprises a mathematical procedure, e.g. transformation carried out on both levels, e.g. calculating logarithm, or other function. In an embodiment comparing comprises making statistics and calculating errors and/or means or averages which is/are considered, and/or assessment of statistics assessment of statistical significance.

A threshold may be an upper limit or a lower limit of a normal range or a typical range which is defined for the purpose of the measurement given.

By “kit” is meant as a collection of materials, comprising a multiplicity of substances in multiple containers, intended to be used in a method, preferably the method (assay) of the invention. Preferably the kit comprises instruction to carry out the method. By “instructions” is meant a list of steps, or a description of the invention, intended to instruct the user, e.g. a practitioner, e.g., a laboratory clinician or technician, to conduct an assay of the invention. The instructions can be written either on paper or in silico, oral (e.g., on an audio medium), or visual (e.g., on a video medium) or in the form of a link to a website.

By a “biological sample”, is meant a sample comprising living immune cells obtained from a mammal and optionally processed. The biological sample can be isolated from the mammal as a body fluid, preferably blood or synovial fluid. Preferably the biological sample is a blood sample. Preferred immune cells are at least T-lymphocytes and/or T lymphocyte subsets and optionally or additionally B-cells.

A “subject” is an individual of a vertebrate, preferably a mammalian species, in particular a primate, a hominid or human.

A “patient” is a subject who is or intended to be under medical or veterinarian care, i.e. observation, supervision, diagnosis or treatment of a condition. Preferably the patient is a primate, a hominid or a human.

A “treatment” refers to any process, action, application, therapy, or the like, wherein the subject or patient is under aid, in particular, medical, or veterinarian aid with the object of improving the subject's or patient's condition, either directly or indirectly.

A “therapy” is understood herein as a method for treatment in which a given medicament or pharmaceutical composition is administered to said patient, preferably administered for a certain period of time with the object of improving the subject's or patient's condition.

A “disease modifying drug” as used herein is a medicament useful to treat IBD.

As in IBD partly the same active agents are used as in rheumatoid arthritis, wherein these compound are effective in general to immune processes, like division of immune cells in case of methothrexate or thiopurines, and reduce inflammation, the “DMARD” terminology is also used in the present application, however, for compounds having an anti-IBD effect. Of note, there are differences in the scope of compounds or their use. For example 5-ASA is often used in IBD but rarely in RA, but the scope is overlapping and the terminology is used.

Thus, a “sDMARD” is a “synthetic disease-modifying antirheumatic drug”, which is, as used herein, a synthetic chemical compound originally defined by their use in rheumatoid arthritis, however, used herein against IBD, to slow down disease progression by targeting the immune system or any immune system pathway and by a mechanism other than lowering inflammation specifically.

In an embodiment csDMARD are different from NSAID (non-steroidal anti-inflammatory drugs).

In a special embodiment NSAID are anti-inflammatory compounds which are act through the inhibition of COX enzymes e.g. Algopyrin, Aspirin, Panadol, Algoflex, Nurofen etc. Preferably NSAIDs are used as a general anti-inflamatory, anti-fever and pain-reducing compounds.

In a sense 5ASA can be considered as an NSAID as to its structure.

In another sense 5ASA is not considered as an NSAID as its is not used as as a general anti-inflammatory, anti-fever and pain-reducing compound.

A “csDMARD” is a “classic synthetic” or “conventional synthetic disease-modifying antirheumatic drug”, preferably with a broad spectrum, which is a synthetic chemical compound defined by their use in rheumatoid arthritis to slow down disease progression by targeting the immune system, typically broadly or in a way not specified yet, and which has been developed not specifically to target JAK inhibition or a specific pathway inside immune cells. Thus, csDMARD are a subgroup of sDMARDs, as described by Smolen, Landewe et al. in the respective EULAR recommendations (Smolen, Landewe et al. 2020).

In a preferred embodiment the csDMARD is selected from the group of compounds consisting of azathioprine, cyclophosphamide (also used in lupus in patients who do not respond to traditional therapy or who experience kidney damage), cyclosporine (used sometimes for lupus in people who do not respond to other therapies), hydroxychloroquine sulfate (an antimalarial drug), leflunomide (people who cannot tolerate methotrexate may take leflunomide. It can also be taken in combination with methotrexate), methotrexate, mycophenolate mofetil (may be used in people whose RA does not respond to other therapies), sulfasalazine (may be used in a triple therapy combination for RA (methotrexate, sulfasalazine, hydroxychloroquine), preferably selected from methotrexate, chloroquine and salazopryne and optionally glucocorticoids.

In a preferred embodiment the definition of “csDMARD” does not involve glucocorticoids.

A “tsDMARD” is a “targeted synthetic disease-modifying antirheumatic drug”, which is a synthetic chemical compound and is anti-inflammatory by reducing or helping the reducing of inflammation by specifically targeting parts of the immune system that play a role in inflammation in the intestine and other organs.

In an embodiment the tsDMARD is a Janus Kinase (JAK) inhibitor. JAK inhibitors have been developed to specifically target the JAK kinase pathway.

Janus kinase inhibitors can be classed in several overlapping classes: they are immunomodulators, they are DMARDs (disease-modifying antirheumatic drugs), and they are a subclass of tyrosine kinase inhibitors. They work by modifying the immune system via cytokine activity inhibition.

Preferably the JAK-inhibitor of the invention is an immunomodulator compound. Preferably the JAK-inhibitor of the invention is inhibitor of JAK1 kinase, preferably selective for JAK1 over JAK2.

In a preferred embodiment the JAK-inhibitor selected from Tofacitinib (e.g. Xeljanz®) and upadacitinib (e.g. Rinvoq®) baricitinib (e.g. Olumiant®).

In an embodiment the tsDMARD is an sphingosine 1-phosphate (S1P) receptor modulator.

In a preferred embodiment the S1P receptor modulator is Ozanimod (Zeposia®), an oral small molecule drug FDA-approved for the treatment of moderate-to-severe ulcerative colitis in adults. S1P receptor modulators work by preventing the migration of immune cells from the lymph nodes to the intestines where they cause inflammation in ulcerative colitis.

In a preferred embodiment the tsDMARD is selected from the group consisting of baricitinib, and tofacitinib (Xeljanz).

A “low molecular weight compound”, preferably an anti-IBD compound, is typically a compound having a molar weight or molecular weight (used interchangeably herein) lower than 1000 Da, preferably 900 Da which can be prepared by chemical synthesis, e.g. total synthesis or semi-total synthesis. In a preferred embodiment a low molecular weight compound is understood as a compound which can penetrate into the cell through the cell membrane because of intrinsic lipophilicity of the molecule without the use of cell surface receptors, in particular wherein it has a molecular weight lower than 1000 Da, preferably 900 Da.

“Switch” of a therapy means an alteration of the therapy which comprises the application of a medicament or pharmaceutical composition which has not been applied previously in the patient under care.

In an embodiment it involves the parallel abandonment of the medicament or pharmaceutical composition administered previously to said patient. In another embodiment it involves the continued administration of the medicament or pharmaceutical composition administered previously to said patient, either in a modified or in an unmodified doses or regime. Preferably the condition is IBD, preferably UC.

In particular, a “switch of the therapy” or an “alteration of the therapy”, as used herein, relates to a switch from an initial therapy of IBD, preferably UC, optionally from an initial anti-inflammatory agent, and comprises the modification of a therapy with a DMARD different from the initial agent.

In an embodiment the initial anti-inflammatory agent is a steroid.

In an embodiment the initial anti-inflammatory agent is a cDMARD.

In an embodiment the initial anti-inflammatory agent is a csDMARD.

In an embodiment the DMARD different from the initial agent is csDMARD wherein the initial agent is different therefrom.

In an embodiment the DMARD different from the initial agent is a bDMARD.

In an embodiment the DMARD different from the initial agent is tsDMARD.

In an embodiment the DMARD different from the initial agent is a csDMARD.

In an embodiment the DMARD different from the initial agent is a tsDMARD.

“Predicting the responsiveness” of a therapy (or alternatively “assessing” the success or outcome of a treatment or therapy” is understood herein as a method, e.g. a diagnostic type method resulting in a quantitative value which predicts whether a given treatment or therapy will be effective to a given patient and thereby may contribute to a decision on the way of treatment or therapy in the future or on the continuation or alteration of it. Assessing normally involves measurement including calculation and preferably involves consideration of the results and/or drawing conclusion.

As used herein the indefinite article, even if used in singular form, “a”, “an”, and if context allows, e.g. in a back-reference the definite article “the”, include plural forms as well; i.e. involves the meaning: “one or more”, unless the context dictates otherwise.

The term “comprises” or “comprising” or “including” are to be construed here as having a non-exhaustive meaning and allow the addition or involvement of further features or method steps or components to anything which comprises the listed features or method steps or components.

The expression “consisting essentially of” or “comprising substantially” is to be understood as consisting of mandatory features or method steps or components listed in a list e.g. in a claim whereas allowing to contain additionally other features or method steps or components which do not materially affect the essential characteristics of the use, method, composition or other subject matter. It is to be understood that “comprises” or “comprising” or “including” can be replaced herein by “consisting essentially of” or “comprising substantially” if so required without addition of new matter.

### Abbreviations

ABC ATP-binding cassette
CD Crohn's disease
CS corticosteroid
cDMARD conventional disease-modifying anti-rheumatic drug
csDMARD conventional synthetic disease-modifying anti-rheumatic drug
DAMP danger-associated molecular patterns
DMARD disease-modifying anti-rheumatic drug
DC dendritic cell
GC glucocorticoid
HBSS Hanks' Balanced Salt Solution
IBD inflammatory bowel disease
IFU Instruction for Use
JAK Janus kinase
MAF multidrug activity factor
MDR multidrug resistance
MTX methotrexate
NSAID non-steroid anti-inflammatory agent
PBMC peripheral blood mononuclear cell
RA rheumatoid arthritis
SMD small molecule drug
tsDMARD targeted synthetic disease-modifying anti-rheumatic drug
UC ulcerative colitis

### BRIEF DESCRIPTION OF THE FIGURES

In FIGS. 1 to 9, results of the first patient cohort treated with conventional disease modifying drugs (cDMARDs and GC) are shown. FIGS. 1 and 2 show composite (MDR1+MRP1) activity, abbreviated MAF_{C}. FIGS. 3 and 4 show MDR1 activity, abbreviated MAF_{MDR1}. FIGS. 5 and 6 show MRP1 activity, abbreviated MAF_{MRP1}. FIGS. 7 and 8 show BCRP activity, abbreviated MAF_{BCRP}.

In FIGS. 10 to 14, results of the second patient cohort treated with targeted synthetic disease modifying drugs (tsDMARDs) are shown.

FIG. 1. MAF_{C} values of IBD patients who received steroid therapy. Moderate elevation of MAF_{C} values of CD3⁺ lymphocytes were demonstrated in case of steroid non-responder patients at the time of diagnosis (Week 0, A) and 8 weeks after starting steroid therapy (B) as compared with responders. In case of responders, 8 weeks after starting steroid therapy a slight MAF_{C} down regulation was detected as compared with Week 0 (C). Opposed to responders, MAF_{C} values showed slight up regulation 8 weeks after starting steroid therapy versus Week 0 (D). At the time of diagnosis, cut-off value was determined with 75% sensitivity and 72.72% specificity (E, AUC=0.6136). At week 8, the cut-off sensitivity was as high as 100% (F, AUC=0.7955). (Bars show average±SEM, * if p≤0.005, Student's unpaired t test). Table 3 contains basic patient data.

FIG. 2. MAF_{C} values of IBD patients who received small molecule drug therapy. MAF_{C} value was slightly increased in case of non-responders as compared with responders before starting small molecule drug therapy (A), which became more pronounced with statistical significance at the time of follow-up (B). In case of responders, small molecule drug therapy had no impact on MAF_{C} values (C), although in case of non-responders at follow up week, significant elevation was determined in MAF_{C} values (D). Cut-off values at Week 0 (E) and at follow-up week (F) was determined with relatively low (66,67%) and with high (100%) sensitivity, respectively and moderate (76.92%) specificity. (Bars show average±SEM, * if p≤0.005, Student's unpaired t test). Table 5 contains basic patient data. Follow-up periods: steroid: 8 weeks; 5-ASA, azathioprine: 12 weeks.

FIG. 3. MDR1 activity values of IBD patients who received steroid therapy. Eight weeks after starting steroid therapy, MAF_{MDR1} value was significantly upregulated in steroid non-responders as compared with responders. At the time of diagnosis, MAF_{MDR1} values were slightly smaller in non-responders as compared with responders (A). Opposed to the time of diagnosis, 8 weeks after starting steroid therapy, a significant up-regulation was demonstrated in case of non-responders as compared with responders (B). In case of responders, steroid therapy resulted in moderate down regulation of MAF_{MDR1} values (C), however, in case of non-responders steroid therapy resulted in up-regulation of MAF_{MDR1} values (D). Interestingly, 8 weeks after starting steroid therapy, cut-off value (F) is remarkably smaller than at the time of diagnosis (E). (Bars show average±SEM, * if p≤0.005, Student's unpaired t test). Table 3 contains basic patient data.

FIG. 4. MDR1 activity values of IBD patients who received small molecule drug therapy. MAF_{MDR1} value of CD3⁺ lymphocytes has strong predictive value for small molecule drug treatment in IBD patients. In case of non-responders, MAF_{MDR1} value was slightly smaller than therapy responders (A). Opposed to week 0, MAF_{MDR1} value was significantly higher in case of non-responders as compared with responders at follow-up (B). Successful therapy resulted in marked down regulation of MAF_{MDR1} value (C), however, in non-responders, MAF_{MDR1} value was significantly up-regulated after small molecule drug treatment as compared with week 0 values (D). Cut-off values of MAF_{MDR1} were determined at week 0 (E) and at follow-up week (F), although cut-off value was much more sensitive and specific during follow-up. (Bars show average±SEM, * if p≤0.005, Student's unpaired t test). Table 5 contains basic patient data. Follow-up periods: steroid: 8 weeks; 5-ASA, azathioprine: 12 weeks.

FIG. 5. MRP1 activity values of IBD patients who received steroid therapy. Steroid treatment had no drastic impact on MAF_{MRP1} values. MAF_{MRP1} values of CD3⁺ lymphocytes were determined at the time of steroid treatment initiation and 8 weeks later. At week 0, MAF_{MRP1} values of non-responders were slightly elevated versus responders (A), however, 8 weeks after starting steroid therapy we did not find any alterations between MAF_{MRP1} values (B). Interestingly, in case of responders, steroid treatment resulted in mild upregulation of MAF_{MRP1} values as compared with week 0 (C). Opposed to responders, steroid therapy resulted in moderate down regulation of MAF_{MRP1} values (D). Cut-off values of MAF_{MRP1} were determined at the time of starting steroid therapy (E) and 8 weeks later (F). The cut-off values were same at both time point, however, the predictive value was more sensitive and specific at week 0 as compared with week 8. (Bars show average±SEM, * if p≤0.005, Student's unpaired t test). Table 3 contains basic patient data.

FIG. 6. MRP1 activity values of IBD patients who received small molecule drug therapy. Small molecule drug treatment had no drastic impact on MAF_{MRP1} values. MAF_{MRP1} values were determined on CD3⁺ lymphocytes of IBD patients at the time of diagnosis and after checkup. At the time of diagnosis, MAF_{MRP1} value of non-responders was slightly elevated as compared with responders (A). In contrary with week 0, at follow-up week a moderate MAF_{MRP1} upregulation was detected as compared with responders (B). In case of responders, small molecule drug treatment resulted in moderate increase of MAF_{MRP1} values (C), however, in case of non-responders, therapy resulted in moderate down regulation of MAF_{MRP1} values (D). The cut-off values of MAF_{MRP1} were determined at week 0 (E) and during follow-up week (F), although the predictive value of week 0 was more sensitive and specific as compared with the follow-up week. (Bars show average±SEM, * if p≤0.005, Student's unpaired t test). Table 5 contains basic patient data. Follow-up periods: steroid: 8 weeks; 5-ASA, azathioprine: 12 weeks.

FIG. 7. BCRP activity values of IBD patients who received steroid therapy. Steroid therapy resulted in the up-regulation of MAF_{BCRP} values in non-responders. At the time of diagnosis, MAF_{BCRP} values of non-responders were remarkably smaller as compared with responders (A), however, 8 weeks after starting steroid treatment, MAF_{BCRP} values of non-responders were higher as compared with responders (B). In case of responders (C), steroid treatment resulted in the decline of MAF_{BCRP} values, although in non-responders (D) an up regulation was detected. Cut-off values of BCRP functional activity was also determined at week 0 (E) and week 8 (F). At week 8, the cut-off sensitivity was as high as 100% (F, AUC=0.7955). (Bars show average±SEM, * if p≤0.005, Student's unpaired t test). Table 3 contains basic patient data.

FIG. 8. BCRP activity values of IBD patients who received small molecule drug therapy. Small molecule drug treatment results in the significant down regulation of MAF_{BCRP} values. At week 0, in case of non-responders, MAF_{BCRP} values were remarkably smaller as compared with responders (A), although at follow-up time, the pattern of MAF_{BCRP} values were completely changed, since MAF_{BCRP} values of non-responders were significantly higher as compared with responders (B). In case of responders, small molecule drug treatment resulted in significant down regulation of MAF_{BCRP} values (C), although in case of non-responders, drastic upregulation was demonstrated without statistical significance (D). Cut-off values before therapy (E) and at follow up (F) were also determined, at week 0 sensitivity and at follow-up week the specificity showed higher value. (Bars show average±SEM, * if p≤0.005, Student's unpaired t test). Table 5 contains basic patient data. Follow-up periods: steroid: 8 weeks; 5-ASA, azathioprine: 12 weeks.

FIG. 9. Activity scores of IBD patients who received small molecule drug treatment. Small molecule drug treatment resulted in down regulation of disease activity scores in responders. Mayo (A, for colitis ulcerosa) and CDAI (B, for Crohn's disease) were recorded at the time of diagnosis (week 0, black bars) and during checkups (follow-up week, red bars). Small molecule drug treatment resulted in the down regulation of Mayo and CDAI scores of patients with favorable therapy response. In case of non-responder colitis ulcerosa patients, the Mayo score was enhanced as compared with responders. On the other hand, Crohn's disease patients with poor treatment response showed drastic down regulation of CDAI scores after therapy. (Bars show average±SEM, * if p≤0.005, Student's unpaired t test). Table 5 contains basic patient data. Follow-up periods: steroid: 8 weeks; 5-ASA, azathioprine: 12 weeks.

FIG. 10. MDR1 activity values, measured in CD3+/CD8+ lymphocytes of IBD patients who received Tofacitinib therapy. Two patient groups were compared: patients treated with tofacitinib vs. patients who are non-responders for tofacitinib. MAF_{MDR1} value of CD8+ lymphocytes has strong predictive value for Tofacitinib treatment in IBD patients. In case of non-responders, MAF_{MDR1} value was significantly smaller than the active patient group (A). Bars show average±SD, * if p≤0.05, unpaired t-test. ROC analysis of MAF_{MDR1} on CD8+ cells were also carried out with the cut-off sensitivity of 88,89% and specificity of 58,82%.

FIG. 11. MDR1 activity values, measured in CD14+ monocytes of IBD patients who received Tofacitinib therapy. Two patient groups were compared: patients treated with tofacitinib vs. patients who are non-responders for tofacitinib. MAF_{MDR1} value of CD14⁺ lymphocytes has strong predictive value for Tofacitinib treatment in IBD patients. In case of non-responders, MAF_{MDR1} value was significantly higher than the active patient group (A). Bars show average±SD, * if p≤0.05, unpaired t-test. ROC analysis of MAF_{MDR1} on CD14⁺ cells were also carried out with the cut-off sensitivity of 100% and specificity of 66.67%.

FIG. 12. MRP1 activity values, measured in CD14⁺ monocytes of IBD patients who received Tofacitinib therapy. Two patient groups are compared: patients treated with tofacitinib vs. patients who are non-responders for tofacitinib. MAF_{MRP1} value of CD14⁺ lymphocytes has strong predictive value for Tofacitinib treatment in IBD patients. In case of non-responders, MAF_{MRP1} value was significantly higher than the active patient group (A). Bars show average±SD, ** if p≤0.005, unpaired t-test. ROC analysis of MAF_{MRP1} on CD14⁺ cells were also carried out with the cut-off sensitivity of 100% and specificity of 80%.

FIG. 13. MDR1 activity values, measured in CD3⁺/CD8⁺ lymphocytes of IBD patients who received Tofacitinib therapy. Two patient groups are compared: tofacitinib-naïve patients vs. patients who are non-responders for tofacitinib. MAF_{MDR1} value of CD8⁺ lymphocytes has strong predictive value for Tofacitinib treatment in IBD patients. In case of non-responders, MAF_{MDR1} value was significantly smaller than the naïve patient group (A). Bars show average±SD, * if p≤0.05, unpaired t-test. ROC analysis of MAF_{MDR1} on CD8⁺ cells were also carried out with the cut-off sensitivity of 77,78% and specificity of 78,57%.

FIG. 14 MRP1 activity values, measured in CD14+ monocytes of IBD patients who received Tofacitinib therapy. Two patient groups are compared: tofacitinib-naïve patients vs. patients who are non-responders for tofacitinib. MAF_{MRP1} value of CD14⁺ lymphocytes has strong predictive value for Tofacitinib treatment in IBD patients. In case of non-responders, MAF_{MRP1} value was significantly higher than the naïve patient group (A). Bars show average±SD, * if p≤0.05, unpaired t-test. ROC analysis of MAF_{MRP1} on CD14⁺ cells were also carried out with the cut-off sensitivity of 100% and specificity of 63.64%.

FIG. 15. Treatment schemes for IBD patients and possibilities to shift form one treatment scheme to an other.

### DETAILED DESCRIPTION OF THE INVENTION

The present inventors have surprisingly found that responsiveness of an IBD patient to an intracellularly acting immunosuppressive agent can be assessed by measuring an ABC-transporter activity level, preferably transport activity level. Also the progress or amelioration of the patient's condition can be monitored by the method of the invention.

In the prior art, no proposal has been made that measuring the transport activity of MDR transporters may be appropriate to provide a prediction on the effectiveness or success of conventional or targeted synthetic (cDMARD or tsDMARD, respectively) therapy for IBD patients. Considering the complexity of multi-layered molecular-level immune dysregulation described in the Background of the invention, involvement of diverse set of immune-competent cells and local tissue cells in disease pathology, neither the exact cell population nor the distinct molecule, structure or analyte is obvious what could fulfil the requirements of an appropriate biomarker. Multidrug transporter activities measured in the present study aiming to investigate the pharmacokinetic interaction properties between well-known multidrug transporter proteins (P-gp/MDR1/ABCB1; MRP1/ABCC1 and BCRP/ABCG2) uncovered unexpectedly a correlation between transporter activity in various mononuclear immune cells (namely CD3+ lymphocytes, or specifically in the CD8+ subtype and CD14+ peripheral monocytes) and therapeutic responsiveness.

These cells have no direct connection with inflammation sites in the bowel and the connection between the transport activity of the multidrug transporter proteins and of the responsiveness of a patient in various anti-IBD therapies at the early phase of the therapy is a highly surprising finding by the present inventors.

Thus, the measurement of transport activity of the multidrug transporters allows an early assessment of responsiveness thereby allowing a shift to a further medication in case of non-responsiveness and thereby potentially making possible to avoid an surgical intervention, more specifically removal of inflamed part or entire colon.

Each of the present measurements have been carried out in an early phase, i.e. within 4 months of the treatment regime concerned: at week 0 and then after a follow-up period, at week 8 or 12, depending on treatment. The difference between a given transporter activity in a non-responder patient and a reference transporter activity in a group of patients which are different, e.g. a treatment naïve group or responder group or e.g. a more general group wherein the disease is active and involves several type of patients however a kind of average value is obtained, and wherein the responder value is different, i.e. outstands from this reference level or background, is indicative or even predictive of the non-responder feature.

Despite certain similarities the observation may differ per transporters and types of medicaments and possibly even the observation weeks.

In the first patient cohort IBD patients (moderate to severe disease; n=35; both with Crohn-disease/CD, n=17, and ulcerative colitis/UC, n=18) were enrolled. The patients were treated with steroid alone or with conventional disease modifying drugs (5-ASA, azathioprine) in combination with the steroid, i.e. systemic steroid (methylprednisolone, 32-64 mg) or topical glucocorticoids (budesonide, 9 mg). In the statistical analysis various steroid types have not been differentiated and were treated as “steroids”. In case of patients on steroid alone a follow-up visit and sample collection was carried out at 8 weeks after the start of the treatment whereas in case of treatment with conventional disease modifying drugs the follow-up period was 12 weeks.

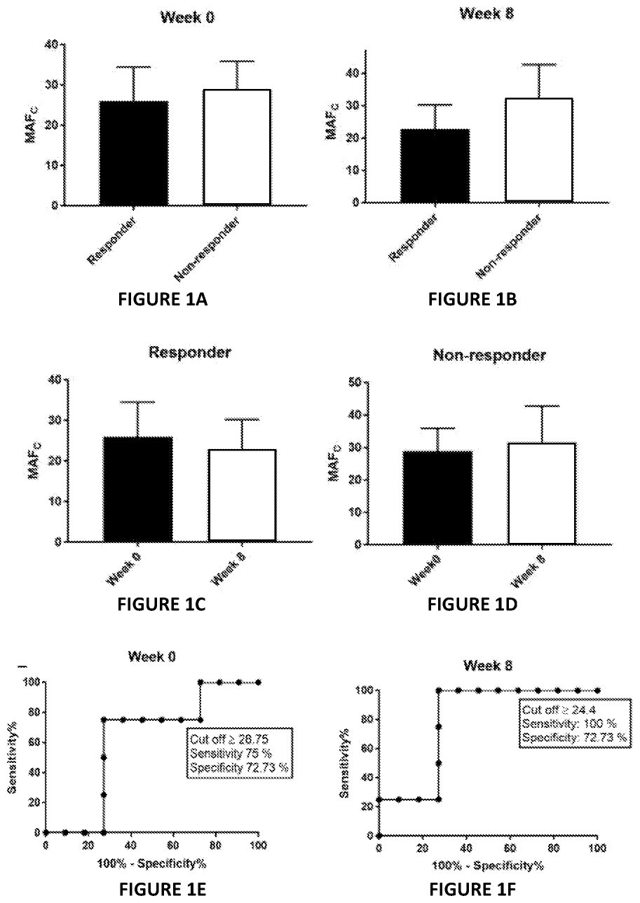

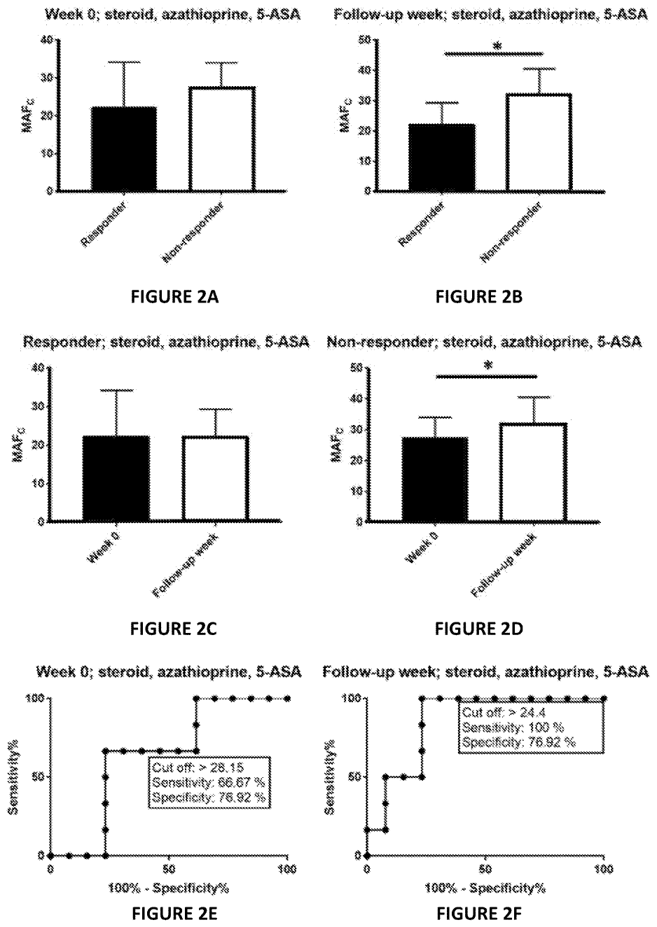

The present inventors were able to show that in case of IBD patients who received steroid therapy, MDR1 and MRP1 transporter activities (shown as MAF_{C} values) were moderately elevated in CD3⁺ lymphocytes in case of steroid non-responder patients already at the time of diagnosis, i.e. at 0 week. Similarly in case of patients receiving steroid, azathioprine, 5-ASA therapy, MAF_{C} value was slightly increased in case of non-responders as compared with responders (see the results shown on FIGS. 1 and 2).

When MAF_{MDR1} value was studied alone in patients on steroid therapy, upregulation was observed later after an 8 weeks follow-up period after diagnosis. (To the contrary, in case of responders, steroid therapy resulted in moderate down regulation of MAF_{MDR1} values.). The results were similar for small molecule drug treatment in IBD patients. Thus, MAF_{MDR1} value of CD3⁺ lymphocytes also has strong predictive value for small molecule drug treatment in IBD patients, in particular after a follow-up period.

MRP1 activity alone showed a relatively small elevation at the time of diagnosis both in patients under steroid treatment and small molecule drug treatment. In case of non-responders, however, MRP1 activity resulted in a down regulation of MAF_{MRP1} values as shown after follow-up.

BCRP activity showed a pronounced down-regulation in non-responders at the time of diagnosis whereas upon follow-up a significant up-regulation was observed in non-responders both in small molecule treated patients and in patients treated by steroids alone.

Thus, diagnostic value of MDR1 activity as well as BCRP activity is inevitable whereas that of MRP1 is less pronounced based on the measurements carried out so far, however as an additional marker may well be useful even based on the present results.

In a second patient cohort, based on the data presented herein and measured with IBD patients who proved to be non-responders to JAK inhibitor (tofacitinib) treatment, the present inventors could show significant differences between the “Active” and “Non-responder” populations in MDR1 transporter activity on CD8+ cells and also in MDR1 and MRP1 activities on CD14+ cells.

In this patient cohort initial and follow-up periods were not differentiated, however, significant changes could be shown even in this mixed samples (samples taken in different times after the initiation of the treatment).

Furthermore, they could show significant differences between the “Naïve” and “Non-responder” populations in MDR1 transporter activity on CD8+ cells and also in MRP1 activity on CD14+ cells.

These results showcase that the transporter activity values, as provided herein by the Theresa kit, could be utilized to determine the efficiency of an ongoing treatment. Measuring the transporter activity from a blood-sample compared to the conventional endoscopy is a less invasive method for the patient and the results are available within a comparatively short timeframe.

ROC curves of significant results were created where the sensitivity-specificity relation is illustrated. While according to the ROC curves the sensitivity of the method regards to the specificity is currently proved to be relatively weak in a part of the measurements. However, the sample number for each comparison is relatively low and the sample size estimates shows that the sensitivity can be considerably raised with the increase of the number of involved patients.

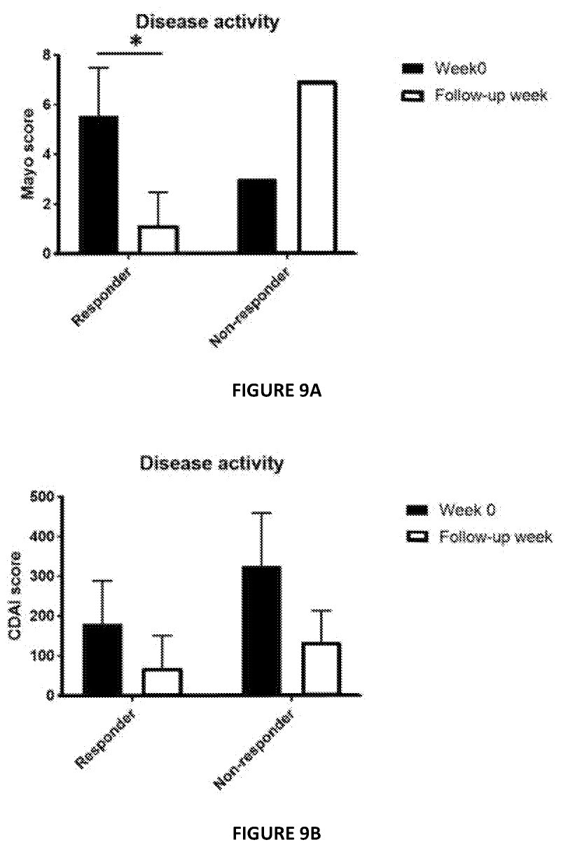

In an alternative embodiment the patient status can be monitored by the method of the invention. As shown on FIG. 9, small molecule drug treatment resulted in down regulation of disease activity scores in responders. Small molecule drug treatment resulted in the down regulation of Mayo and CDAI scores of patients with favorable therapy response. In case of non-responder colitis ulcerosa patients, the Mayo score was enhanced as compared with responders. On the other hand, Crohn's disease patients with poor treatment response showed drastic down regulation of CDAI scores after therapy. Thus, the results support the differentiation between responders and non-responders and can be observed in parallel with ABC-transporter activity measurements as shown on FIGS. 1 to 8.

Based on the result of the method of the invention a decision can be made on the start and/or continuation of the therapy in an initial phase.

For example, without limitation, a decision on switching from an initial glucocorticoid or NSAID therapy to a steroid therapy or to a combined small molecule IBD drug therapy is supported by the present invention wherein the start of a new therapy is supported provided that a responsiveness of the patient is predicted by the method of the invention or the patient is not considered being non-responsive. Thus, once the patient is found responsive to therapy with the combined small molecule IBD drug by the method it can be started or continued.

As a further example, without limitation, a decision on switching from cDMARD therapy to a tsDMARD therapy may be brought provided that a responsiveness of the patient to tsDMARD is predicted by the method of the invention. Alternatively the decision is brought if the patient is not found non-responsive to said tsDMARD.

Further variants are mentioned in the description.

Therapeutic regimes are well known in the art.

For example immunosuppression combined with other therapies are described by D'Haens G et al. (D'Haens G et al. 2008).

Further method of diagnosis and management of Crohn disease and Ulcerative Colitis are well described in consensus papers (Gomollón F et al., 2017, Harbord M. et al., 2022).

Novel therapies of UC are proposed by Kaur A and Goggolidou P (Kaur A and Goggolidou P 2020) as well as Hazel K and O'Connor A (Hazel K and O'Connor A 2020).

Public Assessment Reports of the European Medicines Agency also provide a teaching on various therapies as listed above (see European Medicines Agency Xeljanz Public Assessment Report, EMA/CHMP/853224/2016, Olumiant Public Assessment Report EMA/13493/2017, 15 Dec. 2016, Jyseleca Public Assement Report, EMA/424374/2020, Rinvoq Public Assessment Report, EMA/608624/2019 Corr., Inrebic Public Assessment Report, EMA/705612/2020, Cibingo Public Assessment Report, EMA/647846/2021).

Further examples for alterations of ABC-transporter levels indicating responsiveness are given in the Examples chapter.

### EXAMPLES

### Example 1: Material and Methods

### Patient Treatments

Participating patients were enrolled in the study at University of Szeged Albert Szent-Györgyi Medical School, 1st dept. of Medicine after acquiring written informed consent from all patients. The study was conducted in accordance with the authorization issued by the responsible national regulatory authority (OGYEI, reg Nr 42432-μcohort, IBD patients (moderate to severe disease; n=35; both with Crohn-disease/CD, n=17, and ulcerative colitis/UC, n=18) were enrolled and treated with conventional disease modifying drugs (5-ASA, azathioprine) in combination with systemic (methylprednisolone, 32-64 mg) glucocorticoids.

In a second cohort, patients with severe IBD (UC only) treated with tofacitinib participated. Patients were assigned to treatment groups based on drug therapy administered. Patients in tofacitinib-naïve group did not take a single tofacitinib dose prior to first blood sampling and started tofacitinib treatment on the day of blood draw. Patients were considered on active treatment if they started tofacitinib treatment in the past and were actively taking tofacitinib at the time of blood sampling. This group included patients at their 12-weeks control visit if tofacitinib was not discontinued prematurely for any reason. Patients in non-responder group stopped tofacitinib earlier because of lack of effectiveness and their disease status was still active and severe. Peripheral blood samples anticoagulated with K₃-EDTA were collected from all patients.

### Cells

Human peripheral blood mononuclear cells (PBMCs) were isolated from whole blood of IBD/UC patients according to standard procedures with Ficoll-Hypaque density gradient centrifugation. Blood samples were collected once or twice from each patient in sterile blood collection tubes containing tri-potassium EDTA (Vacuette, Greiner-BioOne). In case of certain patients there was no possibility to carry out a second, follow-up sample collection for example if surgical intervention (i.e. colectomy) was carried out in the interim.

In case of the first cohort, small molecule drug therapy pre-treatment samples typically were collected first when the patient had not started taking drugs yet and once at the expiry follow-up (FU) periods: steroid for 8 weeks, 5-ASA or azathioprine for 12 weeks.

In case of the second cohort, JAK inhibitor samples were collected first, when the patient had not started taking tofacitinib yet and once after 12 weeks of tofacitinib treatment (control visit).

### Theresa Kit Reagents

TABLE 1
Reagents of the Theresa kit.

| | | | Concen- | | | |
|---|---|---|---|---|---|---|
| Reagent | Amount | | tration | | Usage | Function |
| Calcein-AM; | 60 | μl | 121 | μM | should be | fluorogenic |
| Reagent 1. | | | | | diluted | MDR1 and |
| | | | | | | MRP1 substrate |
| Tariquidar; | 180 | μl | 2.02 | μM | ready-to- | selective |
| Reagent 2. | | | | | use | inhibition |
| | | | | | | of MDR1 |
| Indomethacin; | 180 | μl | 2.53 | mM | ready-to- | selective |
| Reagent 3. | | | | | use | inhibition |
| | | | | | | of MRP1 |
| Phen Green- | 60 | μl | 211.4 | μM | should be | fluorogenic |
| SK-diacetate; | | | | | diluted | BCRP |
| Reagent 4. | | | | | | substrate |
| KO143- | 180 | μl | 101 | μM | ready-to- | selective |
| hydrate; | | | | | use | inhibition |
| Reagent 5. | | | | | | of BCRP |
| K3-EDTA; | 550 | μl | 40 | mM | should be | base of reaction |
| Reagent 6. | | | | | diluted | buffer, vehicle |
| | | | | | | of substrates |

### Antibodies

To measure MDR activity on separated cell populations fluorophore conjugated population marker antibodies were used after Theresa protocol (see Table 1).

TABLE 2
Antibodies used in the measurements.

| | Conjugated | Manu- | Amount per | | Incubation time/ |
|---|---|---|---|---|---|
| Antibody | fluorophore | facturer | sample | | temperature |
| Anti-human | PE-Cyanine7 | Biolegend | 1 | μl | 15 minutes/on ice |
| CD14 | | | | | |
| Anti-human | PE-Cyanine7 | Biolegend | 1 | μl | 15 minutes/on ice |
| CD4 | | | | | |
| Anti-human | PE-Cyanine7 | Biolegend | 1.5 | μl | 15 minutes/on ice |
| CD8 | | | | | |

### Flow Cytometry for Steroid Treated IBD Patients

Flow cytometry measurements were performed using Solvo MDQ kit. Reagents were prepared according to standard Instruction for Use leaflet provided with Solvo MDQ kit. For MDR1 activity measurements CD3⁺ lymphocytes were loaded with calcein-AM and transporter activity inhibited with verapamil. For MRP1 activity reagent pair of calcein (fluorescent probe) and indomethacin (MRP1 specific inhibitor), for BCRP activity mitoxantrone (fluorescent probe) and KO-134 (inhibitor) were used, respectively.

Isolated PBMCs were first washed twice with Hanks' Balanced Salt Solution by centrifugation (300 g, 5 min each). Then 5×10⁵ pelleted PBMCs per sample were treated according to IFU. The cell suspensions were kept at 37±5° C. in a water bath before and during the treatment and stored on ice, protected from lights until performing measurements.

### Flow Cytometry Using Theresa Kit

### Preparations

The diluted amounts are calculated for the MDR activity measurement in case of one donor. The following dilutions should be multiplied in case of more than one donor to get the appropriate reagent amounts. The transporter inhibitors are ready-to-use, no dilution required.

To prepare 100 μM K3-EDTA containing HBSS solution 47.5 μl of Reagent 6 stock solution was diluted with 18.95 ml of HBSS solution. The calcein-AM working solution was made by diluting 5 μl of Reagent 1 stock solution with 995 μl of 100 μM EDTA-HBSS working solution. This calcein solution need to be used within 1 hour after the preparation. To have the working solution of Phen Green-SK diacetate, 5 μl of Reagent 4 stock solution was diluted with 695 μl of 100 μM EDTA-HBSS working solution. This Phen Green working solution can be used within 15 minutes after the preparation.

Isolated PBMCs were first washed twice with Hanks' Balanced Salt Solution (HBSS, Capricorn) by centrifugation (300 g, 5 min each). Then 5×10⁵ pelleted PBMCs per sample were resuspended with 500 μl 100 μM Reagent 6-containing HBSS. The cell solutions were kept at 37±5° C. in a water bath before and during the treatment.

Treatment with Theresa Reagents

To block MDR transporters cell suspensions were treated with 5 μl ready-to-use 2.02 UM tariquidar (Reagent 2), 2.53 mM indomethacin (Reagent 3) or 101 μM KO143 (Reagent 5) for 5 minutes at 37±5° C. (for control samples 5 μl 100 μM Reagent 6-containing HBSS was used without inhibitor). Without washing, previously diluted to working concentration, 0.605 μM calcein-AM (Reagent 1) or 1.51 μM Phen Green (Reagent 4) were added (100 μl) to appropriate samples for further 10 minutes at 37±5° C. protected from light followed by an immediate centrifugation at 1000 g for 1 minute. To label the cells with population marker antibodies supernatants were removed. Then samples were placed on ice as soon as possible to minimize MDR transporter activity.

### Antibody Labeling

Primary cells were also stained with anti-human CD14, CD4 or CD8 to discriminate between monocytes, CD4⁺ and CD8⁺ lymphocytes, respectively. Circumstances of Ab labeling is shown in Table 2. After incubation the unbound antibodies were removed with 1 ml ice-cold HBSS without Reagent 6 (centrifugation at 1000 g for 1 minute). Samples were measured on Partec/Sysmex Cyflow Space flow cytometer and analyzed by FlowJo V10 software.

### Statistical Analysis

Statistical analysis was performed using TIBCO's Statistica (version 14.0 for Windows). A p value<0.05 was considered statistically significant. The specific statistical tests applied are indicated in the Brief description of the figures. Patient data collected from patients discontinued early or missed 12-weeks control visit for any reason was left out from analysis (missing longitudinal evaluation).

### Example 2: Results of the First Patient Cohort Treated with Conventional Disease Modifying Drugs (cDMARDs and GC)

In the first patient cohort IBD patients (moderate to severe disease; n=35; both with Crohn-disease/CD, n=17, and ulcerative colitis/UC, n=18) were enrolled and treated with conventional disease modifying drugs (5-ASA, azathioprine) in combination with systemic (methylprednisolone, 32-64 mg) or topical glucocorticoids (budesonide, 9 mg).

### Composite (MDR1 and MRP1) Activity

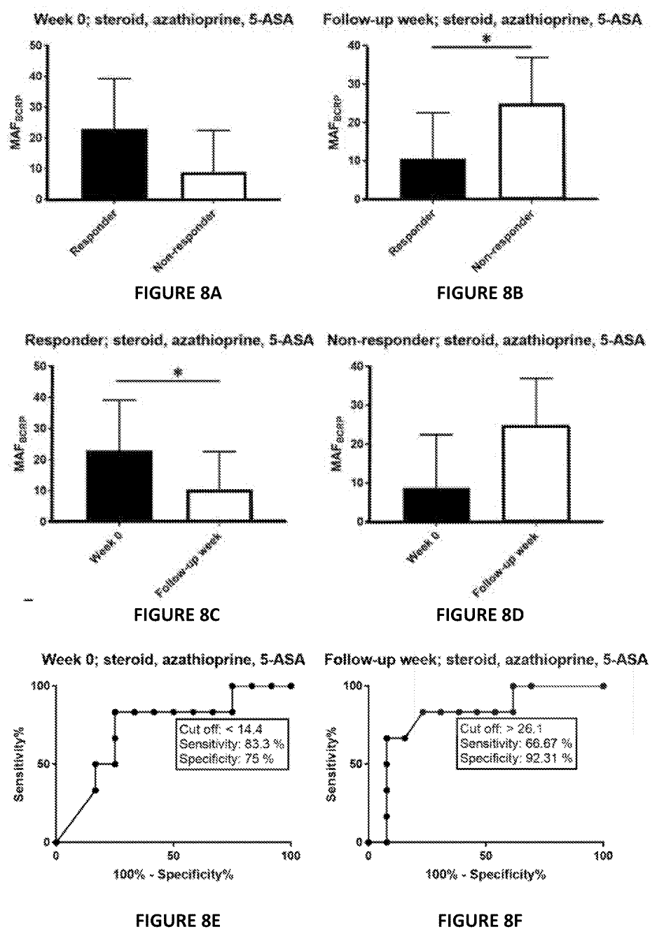

In case of IBD patients who received steroid therapy, moderate elevation of MAF_{C} values of CD3⁺ lymphocytes were demonstrated in case of steroid non-responder patients at the time of diagnosis (Week 0, FIG. 1A) and 8 weeks after starting steroid therapy (FIG. 1B) as compared with responders. In case of responders, 8 weeks after starting steroid therapy a slight MAF_{C} down regulation was detected as compared with Week 0 (FIG. 1C). Opposed to responders, MAF_{C} values showed slight up regulation 8 weeks after starting steroid therapy versus Week 0 (FIG. 1D). At the time of diagnosis, cut-off value was determined with 75% sensitivity and 72.72% specificity (FIG. 1E, AUC=0.6136). At week 8, the cut-off sensitivity was as high as 100% (FIG. 1F, AUC=0.7955).

TABLE 3
Baseline patient data of IBD patient who received steroid treatment

| | |
|---|---|
| Total patient number (men/women) | 15 (11/4) |
| Crohn's disease (men/women) | 7 (6/1) |
| Responder | 3 |
| Non-responder | 4 |
| Colitis ulcerosa (men/women) | 8 (5/3) |
| Responder | 8 |
| Non-responder | 0 |

TABLE 4
Basic laboratory data of patients (steroid)

| | | Responder | | Non-responder | |
|---|---|---|---|---|---|
| CRP (mg/L) | Week 0 | 55.42 | (2-151.2)^{a} | 83.31 | (6.8-166) |
| | Week 8 | 6.1 | (2.9-14.8) | 6.02 | (2.1-13.7) |
| Fe (μmol/L) | Week 0 | 7.77 | (2.1-19.3)^{a} | 9.2 | (4.3-14.1) |
| | Week 8 | 17.15 | (4.1-39.8) | 15.25 | (4.6-26) |
| WBC (G/L) | Week 0 | 11.02 | (5.03-19.73) | 9.42 | (7.69-10.6) |
| | Week 8 | 12.64 | (6.64-24.18) | 12.62 | (7.91-17.28) |
| Htk (%) | Week 0 | 39.5 | (26-46) | 35.5 | (30-42)^{b} |
| | Week 8 | 42.63 | (36-47) | 40.75 | (38-44) |
| Hgb (G/L) | Week 0 | 130.3 | (82-154) | 116.5 | (99-141)^{b} |
| | Week 8 | 137.27 | (116-159) | 132.5 | (126-150) |
| PLT (G/L) | Week 0 | 394.8 | (229-501)^{c} | 478 | (258-880) |
| | Week 8 | 320.9 | (153-407) | 307 | (213-460) |

CRP: C reactive protein,
WBC: white blood cell,
Htk: hematocrite,
Hgb: haemoglobine,
Plt: platelet
^{a}responder week 0 vs. week 8
^{b}non-responder, week 0 vs week 8
^{c}responder vs non-responder, week 0
^{d}responder vs non-responder, week 8, if p ≤ 0.05, Student's paired t test

In case of patients receiving steroid, azathioprine, 5-ASA therapy, MAF_{C} value was slightly increased in case of non-responders as compared with responders before starting small molecule drug therapy (FIG. 2A), which became more pronounced with statistical significance at the time of follow-up (FIG. 2B). In case of responders, small molecule drug therapy had no impact on MAF_{C} values (FIG. 2C), although in case of non-responders at follow up week, significant elevation was determined in MAF_{C} values (FIG. 2D). Cut-off values at Week 0 (FIG. 2E) and at follow-up week (FIG. 2F) was determined with high (100%) specificity and moderate (76.92%) sensitivity.

TABLE 5
Baseline patient data of IBD patients who
received small molecule drug therapy

| Total patient number (men/women) | 19 | (14/5) |
|---|---|---|
| Crohn's disease (men/women) | 9 | (8/1) |
| Responder | 5 | |
| Non-responder | 4 | |
| Colitis ulcerosa (men/women) | 10 | (6/4) |

| | |
|---|---|
| Responder | 9 |
| Non-responder | 1 |

TABLE 6
Basic laboratory parameters of patients (all)

| | | Responder | | Non-responder | |
|---|---|---|---|---|---|
| CRP (mg/L) | Week 0 | 43.11 | (2-151.2)^{a} | 67.17 | (2.6-166) |
| | Week 8 | 5.65 | (2.9-14.8)^{a} | 5.64 | (2.1-13.7) |
| Fe (μmol/L) | Week 0 | 8.71 | (2.1-19.3) | 7.8 | (4.3-14.1) |
| | Week 8 | 17.25 | (4.1-39.8) | 13.66 | (4.6-26) |
| WBC (G/L) | Week 0 | 10.36 | (4.16-19.73) | 8.86 | (6.63-10.6) |
| | Week 8 | 11.38 | (3.8-24.18) | 10.94 | (4.26-17.28) |
| Htk (%) | Week 0 | 40.75 | (26-48)^{c} | 36.2 | (30-42) |
| | Week 8 | 42.92 | (36-47) | 40 | (37-44) |
| Hgb (G/L) | Week 0 | 134.83 | (82-161) | 119 | (99-141) |
| | Week 8 | 139.61 | (116-159) | 130.6 | (123-150) |
| PLT (G/L) | Week 0 | 374.08 | (229-501) | 438.8 | (258-880) |
| | Week 8 | 305.53 | (153-407) | 309.4 | (213-460) |

CRP: C reactive protein,
WBC: white blood cell,
Htk: hematocrite,
Hgb: haemoglobine,
Plt: platelet
^{a}responder week 0 vs. week 8
^{b}non-responder, week 0 vs week 8
^{c}responder vs non-responder, week 0
^{d}responder vs non-responder, week 8, if p ≤ 0.05, Student's paired t test

### MDR1 Activity

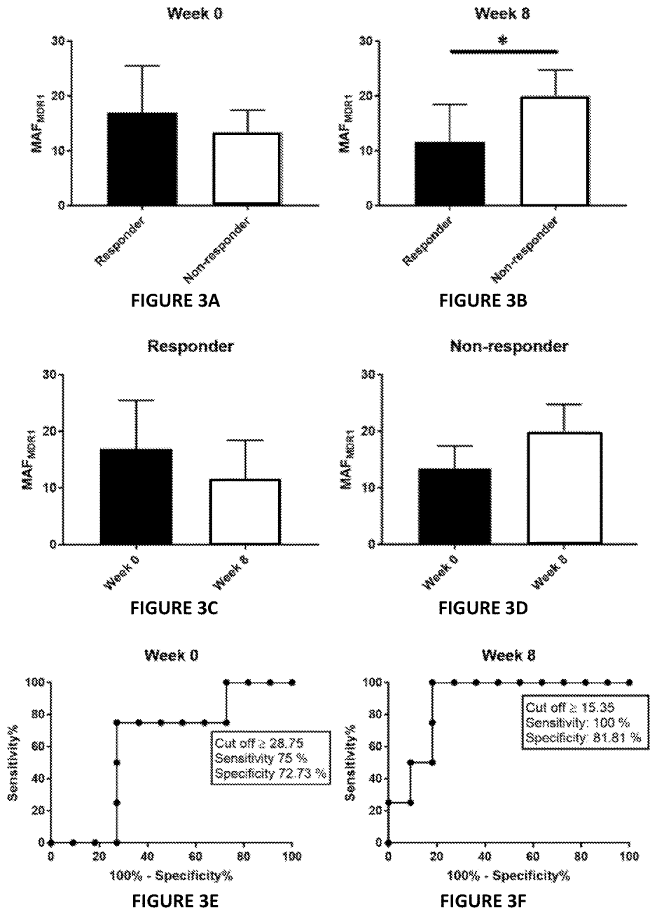

8 weeks after starting steroid therapy, MAF_{MDR1} value was significantly upregulated in steroid non-responders as compared with responders. At the time of diagnosis, MAF_{MDR1} values were slightly smaller in non-responders as compared with responders (FIG. 3A). Opposed to the time of diagnosis, 8 weeks after starting steroid therapy, a significant up-regulation was demonstrated in case of non-responders as compared with responders (FIG. 3B). In case of responders, steroid therapy resulted in moderate down regulation of MAF_{MDR1} values (FIG. 3C), however, in case of non-responders steroid therapy resulted in up-regulation of MAF_{MDR1} values (FIG. 3D). Interestingly, 8 weeks after starting steroid therapy, cut-off value (FIG. 3F) is remarkably smaller than at the time of diagnosis (FIG. 3E).

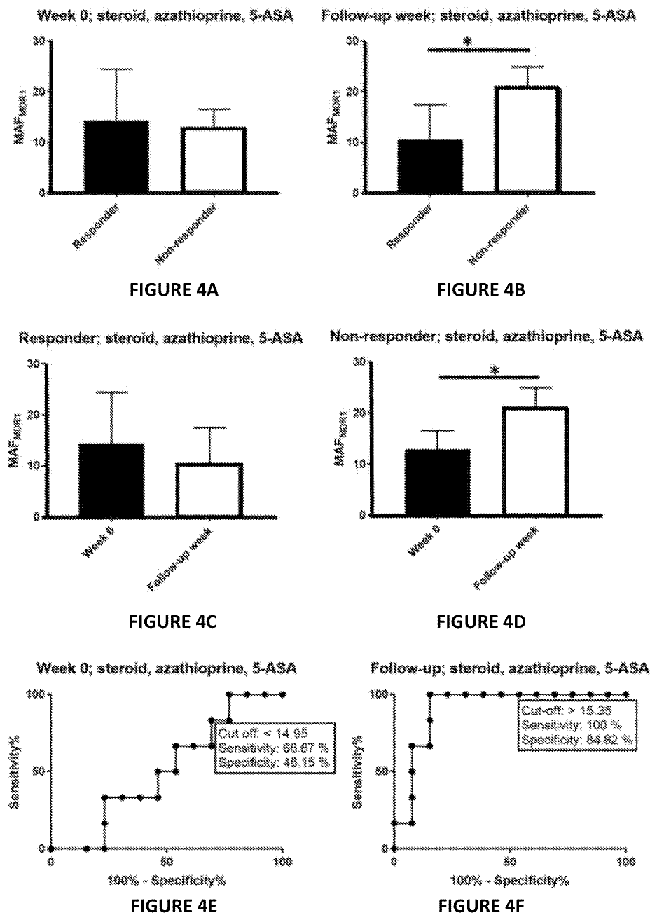

MAF_{MDR1} value of CD3⁺ lymphocytes has strong predictive value for small molecule drug treatment in IBD patients. In case of non-responders, MAF_{MDR1} value was slightly smaller than therapy responders (FIG. 4A). Opposed to week 0, MAF_{MDR1} value was significantly higher in case of non-responders as compared with responders at follow-up (FIG. 4B). Successful therapy resulted in marked down regulation of MAF_{MDR1} value (FIG. 4C), however, in non-responders, MAF_{MDR1} value was significantly up-regulated after small molecule drug treatment as compared with week 0 values (FIG. 4D). Cut-off values of MAF_{MDR1} were determined at week 0 (FIG. 4E) and at follow-up week (FIG. 4F), although cut-off value was much more sensitive and specific during follow-up.

### MRP1 Activity

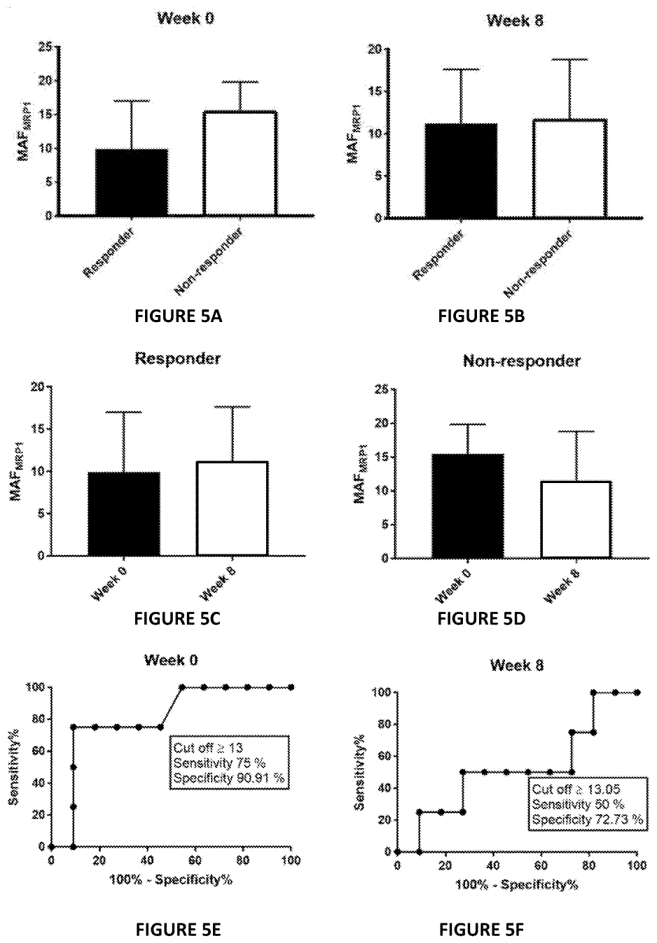

Steroid treatment had some but no drastic impact on MAF_{MRP1} values. MAF_{MRP1} values of CD3⁺ lymphocytes were determined at the time of steroid treatment initiation and 8 weeks later. At week 0, MAF_{MRP1} values of non-responders were slightly elevated versus responders (FIG. 5A), however, 8 weeks after starting steroid therapy we did not find any alterations between MAF_{MRP1} values (FIG. 5B). Interestingly, in case of responders, steroid treatment resulted in mild upregulation of MAF_{MRP1} values as compared with week 0 (FIG. 5C). Opposed to responders, steroid therapy resulted in moderate down regulation of MAF_{MRP1} values (FIG. 5D). Cut-off values of MAF_{MRP1} were determined at the time of starting steroid therapy (FIG. 5E) and 8 weeks later (FIG. 5F). The cut-off values were same at both time point, however, the predictive value was more sensitive and specific at week 0 as compared with week 8.

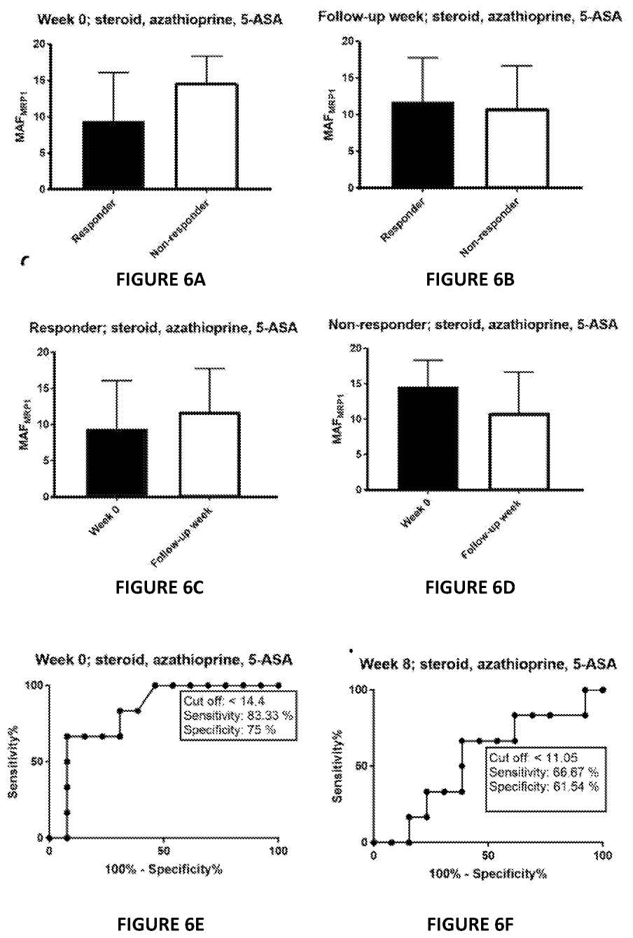

Small molecule drug treatment also had some but no drastic impact on MAF_{MRP1} values. MAF_{MRP1} values were determined on CD3⁺ lymphocytes of IBD patients at the time of diagnosis and after checkup. At the time of diagnosis, MAF_{MRP1} value of non-responders was slightly elevated as compared with responders (FIG. 6A). In contrary with week 0, at follow-up week a moderate MAF_{MRP1} upregulation was detected as compared with responders (FIG. 6B). In case of responders, small molecule drug treatment resulted in moderate increase of MAF_{MRP1} values (FIG. 6C), however, in case of non-responders, therapy resulted in moderate down regulation of MAF_{MRP1} values (FIG. 6D). The cut-off values of MAF_{MRP1} were determined at week 0 (FIG. 6E) and during follow-up week (FIG. 6F), although the predictive value of week 0 was more sensitive and specific as compared with the follow-up week.

### BCRP Activity

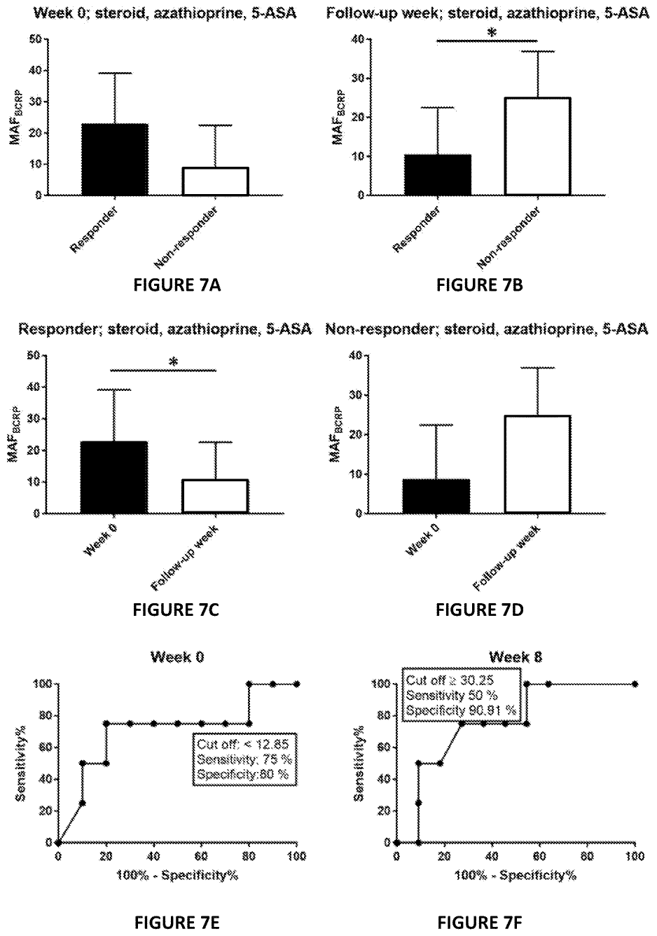

Steroid therapy resulted in the up regulation of MAF_{BCRP} values in non-responders. At the time of diagnosis, MAF_{BCRP} values of non-responders were remarkably smaller as compared with responders (FIG. 7A), however, 8 weeks after starting steroid treatment, MAF_{BCRP} values of non-responders were higher as compared with responders (FIG. 7B). In case of responders (FIG. 7C), steroid treatment resulted in the decline of MAF_{BCRP} values, although in non-responders (FIG. 7D) an up-regulation was detected. Cut-off values of BCRP functional activity was also determined at week 0 (FIG. 7E) and week 8 (FIG. 7F). At week 8, the cut-off sensitivity was as high as 100% (F, AUC=0.7955).

Small molecule drug treatment results in the significant down regulation of MAF_{BCRP} values. At week 0, in case of non-responders, MAF_{BCRP} values were remarkably smaller as compared with responders (FIG. 8A), although at follow-up time, the pattern of MAF_{BCRP} values were completely changed, since MAF_{BCRP} values of non-responders were significantly higher as compared with responders (FIG. 8B). In case of responders, small molecule drug treatment resulted in significant down regulation of MAF_{BCRP} values (FIG. 8C), although in case of non-responders, drastic upregulation was demonstrated without statistical significance (FIG. 8D). Cut-off values before therapy (FIG. 8E) and at follow up (FIG. 8F) were also determined, at week 0 sensitivity and at follow-up week the specificity showed higher value.

### Disease Activity Scores

Small molecule drug treatment resulted in down regulation of disease activity scores in responders. Mayo (FIG. 9A, for colitis ulcerosa) and CDAI (FIG. 9B, for Crohn's disease) were recorded at the time of diagnosis (week 0, black bars) and during checkups (follow-up week, white bars). Small molecule drug treatment resulted in the down regulation of Mayo and CDAI scores of patients with favorable therapy response. In case of non-responder colitis ulcerosa patients, the Mayo score was enhanced as compared with responders. On the other hand, Crohn's disease patients with poor treatment response showed drastic down regulation of CDAI scores after therapy.

### Example 3: Results of the Second Patient Cohort Treated with Targeted Synthetic Disease Modifying Drugs (tsDMARDs)

In second cohort patients with severe IBD (UC only) treated with a JAK inhibitor, i.e. tofacitinib, participated. Patients were assigned to treatment groups based on drug therapy administered. (i) Patients in tofacitinib-naïve group (“Naïve” group) did not take a single tofacitinib dose prior to first blood sampling and started tofacitinib treatment on the day of blood draw. Patients were considered on active treatment (“Active” group) if they started tofacitinib treatment in the past and were actively taking tofacitinib at the time of blood sampling. This group included patients at their 12-weeks control visit if tofacitinib was not discontinued prematurely for any reason. Patients in “Non-responder” group stopped tofacitinib earlier because of lack of effectiveness and their disease status was still active and severe.

Blood samples anticoagulated with K3-EDTA were collected from all patients.

Cell treatment, antibody labeling and flow cytometry using Theresa kit was carried out as described above in the Materials and Methods (Example 1).

Raw measured data are given in tables 7.1, 7.2 and 7.3. below.

Upon statistical analysis the timing of the control and consequent sample collection time was not considered separately due to the relatively small number of patients and because in case of a very few patients only could samples be collected on both occasion (week 0 and at 12 weeks follow-up). Despite this fact, surprisingly valuable markers indicative of responsiveness was found.

TABLE 7.1

| | | Active Patients - On drug and the treatment is effective | | | | | | | | |
|---|---|---|---|---|---|---|---|---|---|---|
| | | CD 14+ | | | CD4+ | | | CD8+ | | |
| Patient | Date of | MAF | MAF | MAF | MAF | MAF | MAF | MAF | MAF | MAF |
| Code | blood test | MDR1 | MRP1 | BCRP | MDR1 | MRP1 | BCRP | MDR1 | MRP1 | BCRP |
| IBDTT010 | 2020 Dec. 14 | | | | | | | 46.7 | 25.15 | |
| IBDTT011 | 2020 Dec. 21 | | | | | | | 35.56 | 23.29 | |
| IBDTT012 | 2021 Jan. 19 | | | | 1.29 | 0.1 | 0.09 | | | |
| IBDTN002 | 2021 Jan. 28 | | | 27.19 | 12.11 | 0.1 | | 67.24 | 22.86 | |
| IBDTN002 | 2021 Dec. 2 | 8.21 | 0.27 | | | | 14.72 | | | 28.27 |
| IBDTT015 | 2021 Mar. 8 | 13.69 | 0.72 | 0.1 | 9.61 | 7.49 | 8.17 | 15.13 | 13.76 | 16.24 |
| IBDTT016 | 2021 Mar. 9 | 0.1 | 0.1 | 0.1 | 4.72 | 0.1 | 0.1 | 25.83 | 13.73 | 22.29 |
| IBDTT021 | 2021 Apr. 1 | 0.1 | 11.78 | 19.96 | 5.03 | 12.96 | 0.96 | 37.64 | 12.85 | 4.87 |
| IBDTN004 | 2021 Apr. 15 | 0.1 | 21.72 | 0.1 | 23.98 | 24.05 | 8.58 | 64.57 | 38.6 | 10.26 |
| IBDTT022 | 2021 Apr. 27 | 22.52 | 10.25 | 0.1 | 15.91 | 30.3 | 11.68 | 28.61 | 41.13 | 20.49 |
| IBDTN003 | 2021 May 4 | 16.24 | 29.87 | 24.54 | 0.1 | 18.27 | 3.57 | 19.03 | 5.68 | 3.39 |
| IBDTN007 | 2021 May 6 | 0.1 | 0.1 | 3.22 | 14.94 | 11.38 | 47.8 | 31.34 | 34.05 | 34.11 |
| IBDTT014 | 2021 May 11 | 10.91 | 11.24 | 0.56 | 9.68 | 27.52 | 15.03 | 26.05 | 48.48 | 21.69 |
| IBDTT002 | 2021 May 17 | 0.1 | 3.97 | 35.1 | 10.57 | 17.88 | 35.44 | 27.02 | 33.23 | 29.23 |
| IBDTT009 | 2021 May 18 | 5.09 | 11.57 | 43.34 | 26.31 | 29.34 | 37.96 | 34.75 | 42/7 | 50.6 |
| IBDTN011 | 2021 May 20 | 0.1 | 0.18 | 0.1 | 0.83 | 2.3 | 0.1 | 319 | 20.84 | 0.86 |
| IBDTT004 | 2021 Jun. 1 | 0.1 | 8.57 | 0.1 | 0.1 | 0.71 | 0.1 | 47.29 | 28.07 | 4.72 |
| IBDTT008 | 2021 Jun. 10 | 7.97 | 0.1 | 18.76 | 14.21 | 8.36 | 24.46 | 44.27 | 36.63 | 12.41 |
| IBDTT003 | 2021 Sep. 28 | 17.69 | 20.15 | 2.94 | 8.08 | 0.1 | 0.1 | 42.84 | 37.59 | 0.1 |

TABLE 7.2

| | | Non-responder patients-Not on drug, the treatment is ineffective | | | | | | | | |
|---|---|---|---|---|---|---|---|---|---|---|
| | | CD14+ | | | CD4+ | | | CD8+ | | |
| Patient | Date of | MAF | MAF | MAF | MAF | MAF | MAF | MAF | MAF | MAF |
| Code | blood test | MDR1 | MRP1 | BCRP | MDR1 | MRP1 | BCRP | MDR1 | MRP1 | BCRP |
| IBDTN001 | 2020 Oct. 8 | | | | 0.1 | 5.8 | | 30.79 | 22.84 | |
| IBDTN001 | 2021 Oct. 12 | 10.02 | 18.05 | 0.1 | | | 1.72 | | | 0.1 |
| IBDTT005 | 2020 Oct. 29 | | | | 16.84 | 16.61 | | 28.95 | 18.38 | |
| IBDTT005 | 2021 Nov. 2 | 18.91 | 20.49 | 4.3 | | | 0.1 | | | 0.1 |
| IBDTT006 | 2021 Jun. 28 | | | | 3.43 | 8.59 | 5.19 | 40.41 | 26.69 | 0.1 |
| IBDTT007 | 2020 Dec. 1 | | | | 14.57 | 13.52 | | 21.95 | 20.24 | |
| IBDTT017 | 2021 May 10 | 18.48 | 42.28 | | 13.87 | 18.78 | 35.51 | 8.39 | 32.08 | 43.54 |
| IBDTT018 | 2021 Jan. 20 | | | | 0.1 | 0.1 | 0.1 | 15.24 | 23.49 | 7.34 |
| IBDTT020 | 2021 Feb. 18 | 15.15 | 36.09 | 35.52 | 0.1 | 7.54 | 0.1 | 28.62 | 35.07 | 0.82 |
| IBDTN009 | 2021 May 3 | 18.88 | 20 | 43.27 | 0.1 | 9.16 | 44.42 | 10.67 | 23.54 | 4.76 |

| | | | | | | | | |
|---|---|---|---|---|---|---|---|---|
| IBDTN013 | 2021 Sep. 14 | no CD14 data | 0.1 | 0.75 | 4.19 | 17.27 | 0.25 | 0.1 |

TABLE 7.3

| | | Naive Patients-No previous drug exposure | | | | | | | | |
|---|---|---|---|---|---|---|---|---|---|---|
| | | CD 14+ | | | CD4+ | | | CD8+ | | |
| Patient | Date of | MAF | MAF | MAF | MAF | MAF | MAF | MAF | MAF | MAF |
| Code | blood test | MDR1 | MRP1 | BCRP | MDR1 | MRP1 | BCRP | MDR1 | MRP1 | BCRP |
| IBDTN001 | 2020 Jun. 23 | | | | | | | | | |
| IBDTN002 | 2020 Sep. 15 | | | | 12.47 | 11.66 | | 53.41 | 16.11 | |
| IBDTN003 | 2020 Oct. 7 | | | | 6.01 | 12.14 | | 8.50 | 6.51 | |
| IBDTN004 | 2020 Dec. 9 | | | | | | | 39.10 | 11.21 | |
| IBDTN005 | 2020 Dec. 23 | 11.70 | 12.78 | | 18.93 | 10.70 | | 38.98 | 17.88 | 2.71 |
| IBDTN007 | 2021 Jan. 14 | 0.1 | 0.1 | | 0.1 | 0.1 | 0.1 | 36.93 | 31.52 | |
| IBDTN009 | 2021 Feb. 8 | 16.79 | 14.5 | 0.1 | | | 0.1 | 30.78 | 24.59 | 27.26 |
| IBDTN011 | 2021 Mar. 1 | 0.1 | 12.19 | 6.94 | 2.2 | 13.21 | 24.19 | 24.71 | 17.1 | 1.65 |
| IBDTN012 | 2021 Apr. 22 | 19.88 | 28.44 | 4.13 | 25.74 | 29.41 | 7.01 | 17.73 | 29.57 | 3.92 |
| IBDTN013 | 2021 May 27 | 0.1 | 0.74 | 0.1 | 0.1 | 19.65 | 30.79 | 40.45 | 21.72 | 4.72 |
| IBDTN014 | 2021 May 31 | 21.75 | 21.38 | 2.45 | 0.1 | 9.38 | 0.1 | 54.17 | 31.08 | 12.92 |
| IBDTN015 | 2021 Aug. 10 | 41 | 21.76 | 0.1 | 0.1 | ′ 2.61 | 13.23 | 43.36 | 38.11 | 0.1 |
| IBDTN016 | 2021 Oct. 21 | 4.89 | 19.43 | 1.64 | 20.93 | 16.89 | 1.6 | 45.94 | 17.27 | 3.96 |
| IBDTN017 | 2022 Mar. 10 | 2.03 | 2.41 | 8.46 | 9.32 | 0.1 | 7.67 | 31.58 | 12.24 | 15.21 |
| IBDTN018 | 2022 May 4 | 5.75 | 0.1 | 28.07 | 10.21 | 12.31 | 18.75 | 30.32 | 31.49 | 27.12 |

Note:
The measured negative values are considered as there is no detectable transporter activity (MAF = 0).

These—in practice—zero values were entered as 0.1 to the program, as STATISTICA can handle these values easier than zero. From a practical point of view, MAF with a value of 0.1 can also be considered as the absence of transporter activity, thus we are not masking the data, the method is applicable.

Based on the data measured with a limited number of IBD patients, the present inventors could show significant differences between the “Active” and “Non-responder” populations in MDR1 transporter activity on CD8+ cells and also in MDR1 and MRP1 activities on CD14+ cells.

This result showcases that transport activity measurement, in particular MDR1 and MRP1 transport activities could be utilized to determine the efficiency of an ongoing treatment. Measuring the transporter activity from a blood-sample compared to the conventional endoscopy is a less invasive method for the patient and the results are available within a comparatively short timeframe.

Furthermore, the present inventors also could show significant differences between the “Naive” and “Non-responder” populations in MDR1 transporter activity on CD8+ cells and also in MRP1 activity on CD14+ cells.

This result also suggests that transport activity measurement, in particular MDR1 and MRP1 transport activities could be used to determine which patient will be “Non-responder” a priori to the treatment.

ROC curves of these results were created where the sensitivity-specificity relation is illustrated. According to the ROC curves the sensitivity of the method regards to the specificity suggest and sample size estimates show that the sensitivity can be considerably raised with the increase of involved patients.

An unforeseen finding, no significant difference could be shown between “Naïve” and “Active” groups in any of the cell types.

Moreover, it has been found that the measurement performed on CD4+ cells are not particularly useful because no significant difference could be recognized amongst the transporter activities when CD4+ cells were got from Responder or Non-responder patients. This supports the conclusion that CD4+ cells—being responsible in vast majority for regulating T-cell driven adaptive, or innate immune cell driven processes—are too heterogenous in function and this cell type cannot be applied for predicting therapy response.

Comparison of Patients Treated with Tofacitinib (“Active”) Vs. Patients Who are Non-Responders for Tofacitinib (“Non-Responders”)

In this set of experiments results in non-responders have been compared with patients treated with tofacitinib; however, active patients have not been split up to smaller groups. MDR1 activity

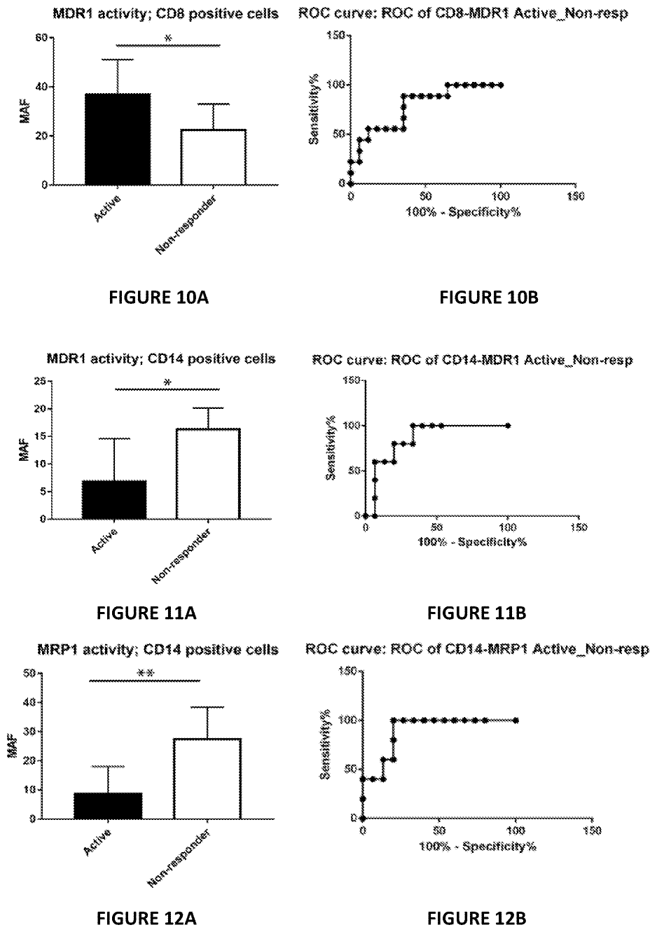

In case of CD3+/CD8+ lymphocytes the measured MAF_{MDR1} value found to have strong predictive value for Tofacitinib treatment in IBD patients. In case of non-responders, MAF_{MDR1} value was significantly smaller than the active patient group (A). (See FIG. 10)

In case of CD14+ monocytes, again, a strong predictive value has been found for Tofacitinib treatment in IBD patients. Here, however, in case of non-responders, MAF_{MDR1} value was significantly higher than the active patient group (A). (See FIG. 11) . . . . .

### MRP1 Activity

In case of CD14+ monocytes MAF_{MRP1} value has proved to be strong predictive for Tofacitinib treatment in IBD patients. In case of non-responders, MAF_{MRP1} value was significantly higher than the active patient group (A), which result is similar as the one obtained for MDR1 in the same group of cells. (See FIG. 12.)

Comparison of Tofacitinib-Naïve Patients Vs. Patients Who are Non-Responders for Tofacitinib

In this set of experiments results in non-responders have been compared with tofacitinib-naïve patients. MDR1 activity

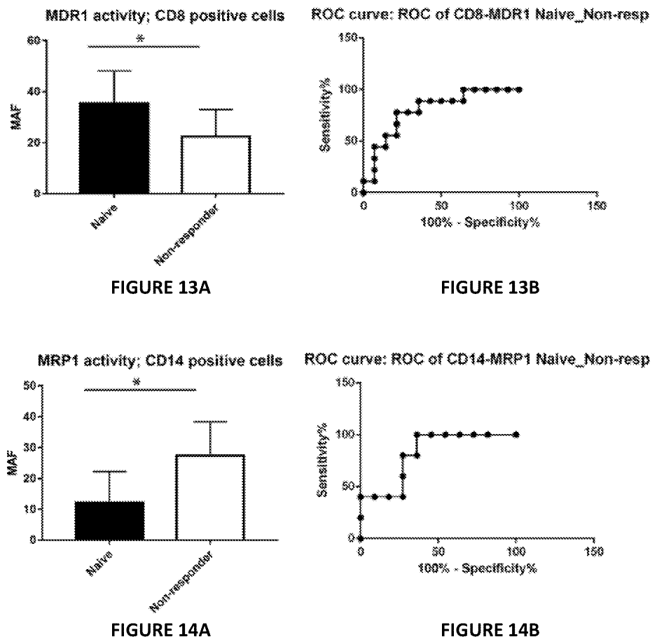

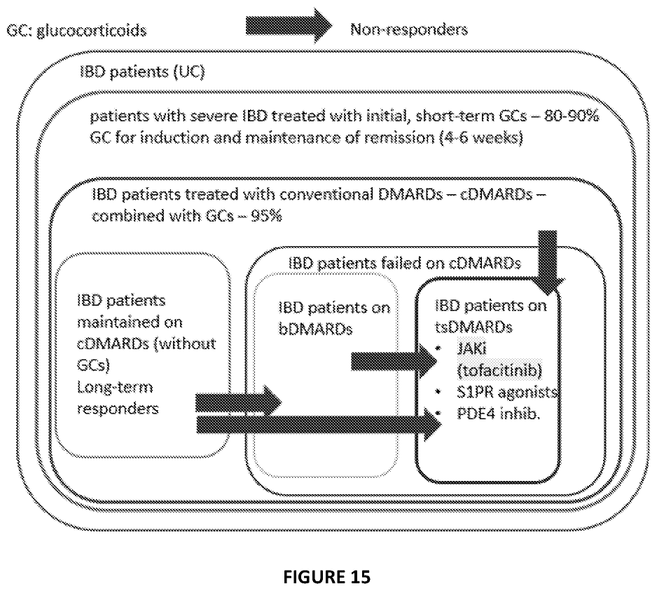

In case of CD3+/CD8+ lymphocytes MAF_{MDR1} value of CD8+ lymphocytes has been found to have a strong predictive value for Tofacitinib treatment in IBD patients. In case of non-responders, MAF_{MDR1} value was significantly smaller than the naive patient group (A). (See FIG. 13.) Thus, the result was analogous to those received for non-responders compared with active patients, indicating that the reference value could be derived from multiple patients groups, for example either from the tofacitinib-naïve patients (which may have been treated previously with other drugs e.g. with steroids or small molecular active agents), or active patients as shown above.

### MRP1 Activity

In case of CD14+ monocytes MAF_{MRP1} value of CD14+ lymphocytes also had strong predictive value for Tofacitinib treatment in IBD patients. In case of non-responders, MAF_{MRP1} value was significantly higher than the naive patient group (A). The result is similar as the one obtained for MDR1 and MRP1 in the same group of cells when non-responders are compared with active patients. (See FIG. 14.)

The above results were also confirmed by our finding that when MAF values for these two transporters were compared in naïve group compared to active group no significant difference was found (data not shown).

### INDUSTRIAL APPLICABILITY

The present inventors have found that responsiveness of an IBD patient to an intracellularly acting immunosuppressive agent can be assessed by measuring an ABC-transporter activity level, preferably transport activity level. Moreover, the progress or amelioration of the patient's condition can be monitored by the method of the invention.

Each of the present measurements presented herein have been carried out in an early phase, i.e. within 4 months of the treatment regime concerned: at week 0 and then after a follow-up period, at week 8 or 12, depending on treatment.

Thus, the present method is useful for supporting decision regarding treatment of IBD and thus is useful in novel and/or improved methods of treatment of IBD.

### REFERENCES

Adegbola, S. O.; Sahnan, K.; Warusavitarne, J.; Hart, A.; Tozer, P. Anti-TNF Therapy in Crohn's Disease. Int. J. Mol. Sci. 2018, 19, 2244. https://doi.org/10.3390/ijms19082244
Bani Ahluwalia, Luiza Moraes, Maria K. Magnusson & Lena Öhman (2018) Immunopathogenesis of inflammatory bowel disease and mechanisms of biological therapies, Scandinavian Journal of Gastroenterology, 53:4, 379-389, DOI: 10.1080/00365521.2018.1447597
Cao Y, Qu C, Chen Y, Li L, Wang X. Association of ABCB1 polymorphisms and ulcerative colitis susceptibility. Int J Clin Exp Pathol. 2015 Jan. 1; 8 (1): 943-7. PMID: 25755800; PMCID: PMC4348806.
Cario, Elke (2017). P-glycoprotein multidrug transporter in inflammatory bowel diseases: More questions than answers. World journal of gastroenterology, 23 (9), 1513-1520. https://doi.org/10.3748/wjg.v23.i9.1513
Chen P, Zhou G, Lin J, Li L, Zeng Z, Chen M, Zhang S. Serum Biomarkers for Inflammatory Bowel Disease. Front Med (Lausanne). 2020 Apr. 22; 7:123. doi: 10.3389/fmed.2020.00123. PMID: 32391365; PMCID: PMC7188783.
Chen, Yan et al. (2022). Comparison of effects of aminosalicylic acid, glucocorticoids and immunosuppressive agents on the expression of multidrug-resistant genes in ulcerative colitis. Scientific Reports 12:20656
D'Haens G, Baert F, van Assche G, et al. Early combined immunosuppression or conventional management in patients with newly diagnosed Crohn's disease: an open randomised trial. Lancet 2008; 371:660-667
De Iudicibus S, Franca R, Martelossi S, Ventura A, Decorti G. Molecular mechanism of glucocorticoid resistance in inflammatory bowel disease. World J Gastroenterol. 2011 Mar. 7; 17 (9): 1095-108. doi: 10.3748/wjg.v17.i9.1095. PMID: 21448414; PMCID: PMC3063901.
de Souza H S, Fiocchi C. Immunopathogenesis of IBD: current state of the art. Nat Rev Gastroenterol Hepatol. 2016; 13:13-27.
Drozdzik et al. (2020). Intestinal drug transporters in pathological states: an overview. Pharmacol. Rep 72, 1173-1194. https://doi.org/10.1007/s43440-020-00139-6
European Medicines Agency, Cibingo Public Assessment Report, EMA/647846/2021, 14 Oct. 2021, (https://www.ema.europa.eu/en/documents/assessment-report/cibingo-epar-public-assessment-report_en.pdf)
European Medicines Agency, Inrebic Public Assessment Report, EMA/705612/2020, 10 Dec. 2020, (https://www.ema.europa.eu/en/documents/assessment-report/rinvoq-epar-public-assessment-report_en.pdf)
European Medicines Agency, Jyseleca Public Assement Report, EMA/424374/2020, 23 Jul. 2020, https://www.ema.europa.eu/en/documents/assessment-report/jyseleca-epar-public-assessment-report_en.pdf
European Medicines Agency, Olumiant Public Assessment Report EMA/13493/2017, 15 Dec. 2016, (https://www.ema.europa.eu/en/documents/assessment-report/olumiant-epar-public-assessment-report_en.pdf)
European Medicines Agency, Rinvoq Public Assessment Report, EMA/608624/2019 Corr. 1, 17 Oct. 2019, (https://www.ema.europa.eu/en/documents/assessment-report/rinvoq-epar-public-assessment-report_en.pdf)
European Medicines Agency, Xeljanz Public Assessment Report, EMA/CHMP/853224/2016, 26 Jan. 2017, (https://www.ema.europa.eu/en/documents/assessment-report/xeljanz-epar-public-assessment-report_en.pdf)
Farrell R J, Murphy A, Long A, Donnelly S, Cherikuri A, O'Toole D, Mahmud N, Keeling P W, Weir D G, Kelleher D. High multidrug resistance (P-glycoprotein 170) expression in inflammatory bowel disease patients who fail medical therapy. Gastroenterology 2000; 118:279-288 [PMID: 10648456 DOI: 10.1016/s0016-5085 (00) 70210-1]
Gomollón F, Dignass A, Annese V, Tilg H, Van Assche G, Lindsay J O, Peyrin-Biroulet L, Cullen G J, Daperno M, Kucharzik T, Rieder F, Almer S, Armuzzi A, Harbord M, Langhorst J, Sans M, Chowers Y, Fiorino G, Juillerat P, Mantzaris G J, Rizzello F, Vavricka S, Gionchetti P; ECCO. 3rd European Evidence-based Consensus on the Diagnosis and Management of Crohn's Disease 2016: Part 1: Diagnosis and Medical Management. J Crohns Colitis. 2017 January; 11 (1): 3-25. doi: 10.1093/ecco-jcc/jjw168. Epub 2016 Sep. 22. PMID: 27660341.
Gottesman, M. M., T. Fojo, and S. E. Bates, Multidrug resistance in cancer: role of ATP-dependent transporters. Nat Rev Cancer, 2002. 2 (1): p. 48-58.
Harbord M, Eliakim R, Bettenworth D, Karmiris K, Katsanos K, Kopylov U, Kucharzik T, Molnár T, Raine T, Sebastian S, de Sousa H T, Dignass A, Carbonnel F; European Crohn's and Colitis Organisation [ECCO]. Third European Evidence-based Consensus on Diagnosis and Management of Ulcerative Colitis. Part 2: Current Management. J Crohns Colitis. 2017 Jul. 1; 11 (7): 769-784. doi: 10.1093/ecco-jcc/jjx009. Erratum in: J Crohns Colitis. 2017 Dec. 4; 11 (12): 1512. Erratum in: J Crohns Colitis. 2022 Aug. 16; PMID: 28513805.
Hayee B, Rahman F Z, Tempero J, McCartney S, Bloom S L, Segal A W, Smith A M. The neutrophil respiratory burst and bacterial digestion in Crohn's disease. Dig Dis Sci 2011; 56:1482-1488.
Hazel K, O'Connor A. Emerging treatments for inflammatory bowel disease. Ther Adv Chronic Dis. 2020 Feb. 5; 11:2040622319899297. doi: 10.1177/2040622319899297. PMID: 32076497; PMCID: PMC7003169.
Kaur A, Goggolidou P. Ulcerative colitis: understanding its cellular pathology could provide insights into novel therapies. J Inflamm (Lond). 2020 Apr. 21; 17:15. doi: 10.1186/s12950-020-00246-4. PMID: 32336953; PMCID: PMC7175540.
Lamas B, Richard M L, Leducq V, Pham H P, Michel M L, Da CostaG, Bridonneau C, Jegou S, Hoffmann T W, Natividad J M, Brot L, Taleb S, Couturier-Maillard A, Nion-Larmurier I, Merabtene F, Seksik P, Bourrier A, Cosnes J, Ryffel B, Beaugerie L, Launay J M, Langella P, Xavier R J, Sokol H (2016) CARD9 impacts colitis by altering gut microbiota metabolism of tryptophan into aryl hydrocarbon receptor ligands. Nat Med 22:598-605
Liefferinckx C, Verstockt B, Gils A, Noman M, Van Kemseke C, Macken E, De Vos M, Van Moerkercke W, Rahier J F, Bossuyt P, Dutre J, Humblet E, Staessen D, Peeters H, Van Hootegem P, Louis E, Franchimont D, Baert F, Vermeire S; Belgian Inflammatory Bowel Disease Research and Development Group [BIRD group]. Long-term Clinical Effectiveness of Ustekinumab in Patients with Crohn's Disease Who Failed Biologic Therapies: A National Cohort Study. J Crohns Colitis. 2019 Oct. 28; 13 (11): 1401-1409. doi: 10.1093/ecco-jcc/jjz080. PMID: 30989232.
Marki-Zay, J., et al., MDR-ABC transporters: biomarkers in rheumatoid arthritis. Clin Exp Rheumatol, 2013. 31 (5): p. 779-87.
Nakase H, Hirano T, Wagatsuma K, Ichimiya T, Yamakawa T, Yokoyama Y, et al. Artificial intelligence-assisted endoscopy changes the definition of mucosal healing in ulcerative colitis. Dig Endosc. (2021) 33:903-11. doi: 10.1111/den.13825
Ng S C, Shi H Y, Hamidi N, Underwood F E, Tang W, Benchimol E I, Panaccione R, Ghosh S, Wu J C Y, Chan F K L, Sung J J Y, Kaplan G G. Worldwide incidence and prevalence of inflammatory bowel disease in the 21st century: a systematic review of population-based studies. Lancet. 2017 Dec. 23; 390 (10114): 2769-2778. doi: 10.1016/S0140-6736 (17) 32448-0. Epub 2017 Oct. 16. Erratum in: Lancet. 2020 Oct. 3; 396 (10256): e56. PMID: 29050646.
Petryszyn, P.; Dudkowiak, R.; Gruca, A.; Ja'zwi'nska-Tarnawska, E.; Ekk-Cierniakowski, P.; Poniewierka, E.; Wiela-Hoje'nska, A.; Glowacka, K. C3435T Polymorphism of the ABCB1 Gene in Polish Patients with Inflammatory Bowel Disease: A Case-Control and Meta-Analysis Study. Genes 2021, 12, 1419.
H. Sales-Campos, H. et al. Classical and recent advances in the treatment of inflammatory bowel diseases Brazilian Journal of Medical and Biological Research (2015) 48 (2): 96-107, http://dx.doi.org/10.1590/1414-431X20143774
Segal A W. Studies on patients establish Crohn's disease as a manifestation of impaired innate immunity. J Intern Med 2019; 286:373-388.
Smolen J S, et al. EULAR recommendations for the management of rheumatoid arthritis with synthetic and biological disease-modifying antirheumatic drugs: 2019 update Ann Rheum Dis 2020, 79, 685-699. doi: 10.1136/annrheumdis-2019-216655
Torres, J., Mehandru, S., Colombel, J.-F., and Peyrin-Biroulet, L. (2017). Crohn's Disease. Lancet 389 (10080), 1741-1755. doi: 10.1016/s0140-6736 (16) 31711-1
Ungaro, R., Mehandru, S., Allen, P. B., Peyrin-Biroulet, L., and Colombel, J.-F. (2017). Ulcerative Colitis. Lancet 389 (10080), 1756-1770. doi: 10.1016/s0140-6736 (16) 32126-2
Vermeire S. Laboratory Markers in IBD: Useful, Magic, or Unnecessary Toys? Gut. (2006) 55:426-31. 10.1136/gut.2005.069476
Weisshof, R.; Golan, M. A.; Yvellez, O. V.; Rubin, D. T. The use of tofacitinib in the treatment of inflammatory bowel disease. Immunotherapy 2018, 10, 837-849.
Wysoczanski R, Kendall A C, Motwani M, Vega R, Rahman F Z, McCartney S, Bloom S L, Nicolaou A, Gilroy D W, Segal A W, Marks D J B. Ulcerative colitis is characterized by amplified acute inflammation with delayed resolution. bioRxiv 2019. https://doi.org/10.1101/870139.
Xu X R, Liu C Q, Feng B S, et al. Dysregulation of mucosal immune response in pathogenesis of inflammatory bowel disease. World J Gastroenterol. 2014; 20:3255-3264.
Zenlea T, Peppercorn M A. Immunosuppressive therapies for inflammatory bowel disease. World J Gastroenterol 2014; 20 (12): 3146-3152 DOI: http://dx.doi.org/10.3748/wjg.v20.i12.3146

1. An in vitro diagnostic method for predicting the responsiveness of an IBD (Irritable Bowel Disease) patient to a therapy with an intracellularly acting immunosuppressive agent of interest, said method comprising the steps of
providing a sample of said IBD patient from a biological sample taken from said IBD patient at a time before treatment with the intracellularly acting immunosuppressive agent of interest or up to the 4^{th} month of treatment with the immunosuppressive agent of interest,
said sample comprising effector mononuclear cells,
obtaining a transporter activity level by measuring transport activity by a multidrug ABC transporter, in the effector mononuclear cells in said sample, and
comparing the transporter activity level with a reference transporter activity level,
said reference transporter activity value being determined in a group of patients, which serve as a reference, i.e. for comparison by
providing biological samples of said group of patients, said sample comprising effector mononuclear cells from said patient, and
obtaining one or more transporter activity value(s) by measuring transport activity by one or more multidrug ABC transporter(s) selected from the group consisting of B, C and G class, in the effector mononuclear cells in said sample, wherein said transporter is the same as the transporter the activity is measured in the method of the invention,
considering said patient as a non-responder to the therapy with the intracellularly acting immunosuppressive agent of interest when the transporter activity level is significantly different from the respective reference transporter activity level
wherein the intracellularly acting immunosuppressive agent is different from a steroid when the multidrug ABC transporter(s) is MDR1.

2. The in vitro method of claim 1 for assessing the status of the IBD patient treated with a previous intracellularly acting immunosuppressive agent, wherein
the intracellularly acting immunosuppressive agent is small molecule drug used in IBD, said small molecule drug being selected from the group consisting of
a glucocorticoid, combined with conventional disease modifying drug, e.g. a DMARD (cDMARD),
a conventional disease modifying drug (cDMARD) without a glucocorticoid,
a non-steroid anti-inflammatory agent (NSAID), and
a targeted synthetic disease-modifying antirheumatic drug (tsDMARD), and the multidrug ABC transporter, selected from the group consisting of B, C and G class of ABC transporters.

3. The in vitro method according to any of claim 1, wherein the biological sample is blood-derived sample.

4. The in vitro method according to claim 3, wherein the effector mononuclear cells are selected from the group consisting of lymphocytes and monocytes.

5. The in vitro method according to claim 1, wherein
the ABC transporters are selected from the group consisting of B, C and G class.

6. (canceled)

7. (canceled)

8. The in vitro method according to claim 1, wherein the effector mononuclear cells are CD3+T-lymphocytes.

9. (canceled)

10. (canceled)

11. The in vitro method according to claim 1, wherein the intracellularly acting immunosuppressive agent of interest is a tsDMARD.

12. The in vitro method according to claim 11, wherein said tsDMARD is a JAK inhibitor.

13. The in vitro method according to claim 2, wherein the ABC transporter is selected from ABCB1 (MDR1), ABCC1 (MRP1) and ABCG2 (BCRP).

14. The in vitro method according to claim 1, wherein the patient having been treated for a given period of time by the intracellularly acting immunosuppressive agent of interest, wherein said period being selected from the group consisting of at least 2 weeks, 4 weeks, 1 month, 2 months and 3 months.

15. (canceled)

16. The in vitro method of claim 12, wherein
the cells are CD3+/CD8+ lymphocytes and MDR1 transport activity is measured in said sample, to obtain MDR1 transport activity value(s), said method comprising
comparing said MDR1 transport activity value(s) with one or more reference values obtained from patients having a low inflammatory status,
considering said patient as a non-responder to the said JAK inhibitor therapy if the MDR1 transport activity values obtained from said sample are significantly lower than said one or more reference values.

17. The in vitro diagnostic method of claim 12, wherein
the cells are monocytes, preferably CD14+ monocytes and MDR1 transport activity is measured in said sample, to obtain MDR1 transport activity value(s)
comparing said MDR1 transport activity value(s) with one or more reference values, said reference value(s) being or having been obtained from patients having a low inflammatory status,
considering said patient as a non-responder to the said JAK inhibitor therapy if the MDR1 transport activity values obtained from said sample are significantly higher than said one or more reference values.

18. The in vitro diagnostic method of claim 12, wherein
the cells are monocytes, preferably CD14+ monocytes and MRP1 transport activity is measured in said sample, to obtain MRP1 transport activity value(s)
comparing said MRP1 transport activity value(s) with one or more reference values
said reference value(s) being or having been obtained from patients having a low inflammatory status,
considering said patient as a non-responder to the said JAK inhibitor therapy if the MRP1 transport activity values obtained from said sample are significantly higher than said one or more reference values.

19. The in vitro diagnostic method of claim 12, wherein said JAK inhibitor is tofacitinib.

20. (canceled)

21. (canceled)

22. The in vitro diagnostic method of claim 1, wherein the patient is or before starting the treatment with it, has been naïve for the intracellularly acting immunosuppressive agent of interest.

23. (canceled)

24. The in vitro diagnostic method of claim 2, wherein
the IBD patients are treated with a small molecule anti-IBD drug as the intracellularly acting immunosuppressive agent of interest,
the cells are lymphocytes, preferably CD3+ lymphocytes and composite MDR1+MRP1 transport activity is measured in said sample, to obtain composite MDR1+MRP1 transport activity values
comparing said composite MDR1+MRP1 transport activity values with one or more reference values
said reference values being or having been obtained from patients having a low inflammatory status,
considering said patient as a non-responder to the said steroid therapy if the composite MDR1+MRP1 transport activity values obtained from said sample are significantly higher than said one or more reference values,
wherein the small molecule anti-IBD drug is selected from steroids, azathioprine and 5-ASA.

25. (canceled)

26. The in vitro diagnostic method of claim 2, wherein
the IBD patients are patients treated with small molecule anti-IBD drug as the intracellularly acting immunosuppressive agent of interest,
MDR1 transport activity is measured in said sample, to obtain MDR1 transport activity value
comparing said MDR1 transport activity values with one or more reference values
said reference values being or having been obtained from patients having a low inflammatory status,
considering said patient as a non-responder to the said steroid therapy if the MDR1 transport activity values obtained from said sample are significantly higher than said one or more reference values;
wherein the small molecule anti-IBD drug is selected from azathioprine and 5-ASA.

27. (canceled)

28. The in vitro diagnostic method of claim 2, wherein
BCRP transport activity is measured in said sample, to obtain BCRP transport activity value
comparing said BCRP transport activity values with one or more reference values
said reference values being or having been obtained from patients having a low inflammatory status,
considering said patient as a non-responder to the said small molecule anti-IBD drug therapy if the BCRP transport activity values obtained from said sample are significantly lower or higher than said one or more reference values,
considering said patient as a responder to said small molecule anti-IBD drug therapy if the BCRP transport activity values obtained from said sample are lower than a reference value obtained for non-responders of the same therapy and/or
considering said patient as a non-responder to said small molecule anti-IBD drug therapy if the BCRP transport activity values obtained from said sample are higher than a reference value obtained for responders of the same therapy, wherein transport activity is measured at 1 to 3 months from the starting of said small molecule treatment
wherein the small molecule anti-IBD drug is selected from azathioprine and 5-ASA.

29. (canceled)

30. A method for treatment of an IBD patient, by predicting the responsiveness of said patient to a therapy with an intracellularly acting immunosuppressive agent of interest, said method comprising the steps of claim 1, said method additionally comprising
carrying out or continuing the therapy with the intracellularly acting immunosuppressive agent of interest,
provided that the patient is
not found non-responsive by the method of the invention to said agent of interest, or
is found responsive by the method of the invention to said agent of interest.
